(19) United States
(12) Patent Application Publication
(10) Pub. No.: US 2023/0133292 A1
(43) Pub. Date: May 4, 2023

(54) HIGH-EFFICIENCY GOLD RECOVERY WITH CUCURBIT[6]URIL

(71) Applicant: Northwestern University, Evanston, IL (US)

(72) Inventors: Huang Wu, Evanston, IL (US); James Fraser Stoddart, Evanston, IL (US)

(21) Appl. No.: 17/907,507

(22) PCT Filed: Mar. 29, 2021
(86) PCT No.: PCT/US21/24612

§ 371 Date: Sep. 27, 2022

Related U.S. Application Data
(60) Provisional application No. 63/000,564, filed on Mar. 27, 2020.

(51) Int. Cl. C22B 11/00; C22B 7/00; C22B 3/10; C22B 3/44

(52) U.S. Cl. CPC C22B 11/04; C22B 7/007; C22B 3/10; C22B 3/44

(57) ABSTRACT

Adducts, superstructures, and crystalline compositions prepared from a metal halide anion non-covalently bound to the outer surface of a macrocycle and methods for gold recovery are disclosed.

### CROSS-REFERENCE TO RELATED APPLICATIONS

The present application claims benefit of priority to U.S. application Ser. No. 63/000,564, filed Mar. 27, 2020, the contents of which is incorporated by reference in its entirety.

### STATEMENT REGARDING FEDERALLY SPONSORED RESEARCH

This invention was made with government support under CHE-1925708 awarded by National Science Foundation. The government has certain rights in the invention.

### BACKGROUND OF THE INVENTION

Developing environmentally benign, highly efficient and thoroughly selective processes for gold recovery is urgently desired for maintaining a sustainable ecologically environment. Gold, on account of its good electrical conductivity, high stability, and excellent malleability, plays an indispensable role in the electronics industry.[4] Nowadays, over 300 tons of gold is used in the electronics every year, accounting for 12% of the annual production of gold from all over the world.[5] Hence, the recovery of gold from e-wastes is extremely important from economic as well as environmental perspective. For well over a century, 83% of gold production depended on a cyanide leaching process, [6] in which elemental gold is convert into the water soluble [Au(CN)₂]⁻, while that remaining is often treated with mercury[7]. Both cyanide and mercury, however, are highly toxic chemicals, [8]which cause human health hazards and serious environmental pollution from inadequate handling and accidental leakages. [7] It is necessary, therefore, to develop an environmentally friendly and sustainable gold recovery process.

### BRIEF SUMMARY OF THE INVENTION

Disclosed herein are high-efficiency gold recovery methods utilizing cucurbiturial. One aspect of the technology includes adducts comprising a metal halide anion non-covalently bound to the outer surface of a macrocycle. In some embodiments, the macrocycle is cucurbitu[6]ril and/or the metal halide anion is [AuX₄]⁻ and X is a halogen.

Another aspect of the technology includes superstructures comprising any of the adducts disclosed herein. In some embodiments, the metal halide anion comprises Cl and the superstructure comprises an alternating one-dimensional supramolecular assembly where adjacent macrocycle are connected to two parallelly aligned metal halide anions. In particular embodiments, the superstructure comprises parallelly aligned one-dimensional supramolecular assemblies. In some embodiments, the metal halide anion comprises Br, the superstructure comprises a two-dimensional supramolecular assembly comprising the macrocycle, and the metal halide anion is accommodated between the lattice space between the two-dimensional supramolecular assemblies.

Another aspect of the technology includes crystalline compositions comprising any of the adducts disclosed herein.

Another aspect of the invention includes method for isolating gold from a gold-bearing material. The method may comprise contacting the gold-bearing material with a hydrogen halide to form a gold-halide solution, contacting the gold-halide solution with a macrocycle to form a precipitate, and isolating the precipitate. The precipitate may comprise any of the adducts, superstructures, or crystalline compositions described herein. The method may further comprise reducing gold of the precipitate with a reductant and, optionally, isolating the reduced gold of the precipitate. In some embodiments, the method further comprises isolating the macrocycle after formation of the adduct and, optionally, isolated macrocycle may be recycled by contacting the isolated macrocycle with the gold-halide solution.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Formation of co-precipitates of CB[6].MAuX₄(M=H/K, X=Cl/Br) from CB[6]and MAuX₄. When CB [6](8 mM, 1.5 mL) in an aqueous HCl (3 M) or HBr (3.5 M) solution is added to aqueous solutions of MAuX₄ (20 mM, 0.6 mL), yellow or brown co-precipitates are formed immediately.

FIG. 2. Gold recovery efficiencies based on the co-precipitates from four aqueous solutions of CB[6] (5.7 mM) and MAuX₄ (5.7 mM) (M=H/K, X=Cl/Br), which are calculated according to the initial and residual concentrations of [AuX₄]⁻ anions in the aqueous solutions. The initial and residual concentrations of [AuX₄]⁻ anions in aqueous solutions were measured at 25° C. by ICP-OES analysis.

FIGS. 3A-3D. Effect of changes in concentration of CB[6] and HAuCl₄ (FIG. 3A), KAuCl₄ (FIG. 3B), HAuBr₄ (FIG. 3C), KAuBr₄ (FIG. 3D) on gold recovery efficiency from the four co-precipitated adducts. The concentration of HCl is 2 M in all aqueous solutions of CB[6].MAuCl₄, while the concentration of HBr is 2.5 M in all aqueous solutions of CB[6].MAuBr₄.

FIGS. 4A-4B. Effect of changes in the concentration of HCl on gold recovery efficiency from two co-precipitated adducts CB[6].HAuCl₄ (FIG. 4A) and CB[6].KAuCl₄ (FIG. 4B). The concentrations of CB[6] and MAuCl₄ (M=H or K) in all aqueous solutions are 6 mM.

FIGS. 5A-5D. Solid-state superstructure of the adduct formed between CB[6] and HAuCl₄. (FIG. 5A) Ball-and-stick representation showing that every CB[6] molecule interacts with four [AuCl₄]⁻ anions through [Au—Cl . . . H—C] hydrogen bonding and ion-dipole interactions. (FIG. 5B) The supramolecular assembly of the one-dimensional nanostructure extending along the b-axis in which adjacent CB[6] molecules are connected by two parallelly aligned [AuCl₄]⁻ anions.

(FIG. 5C) Structural formulas of CB[6] and [AuCl₄]⁻ anion. (FIG. 5D) Solid-state superstructure of CB[6].HAuCl₄, which is made up of the parallelly aligned one-dimensional supramolecular assembly illustrated in (FIG. 5B). The H₂O molecules are omitted for the sake of clarity. H gray, C pale blue, N blue, O red, Cl green, Au yellow.

FIGS. 5E-5H Solid-state superstructures of the adduct formed between CB[6] and KAuCl₄. (FIGS. 5E and 5G) Ball-and-stick representations showing different views of every CB[6] interacting with four [AuCl₄]⁻ anions through [Au—Cl . . . H—C] hydrogen bonding and ion-dipole interactions. (FIG. 5F) The supramolecular assembly of the one-dimensional nanostructure, extending along the b-axis in which adjacent CB[6] molecules are connected by two parallelly aligned [AuCl₄]⁻ anions, is portrayed. (FIG. 5H) The solid-state superstructure of CB[6].HAuCl₄ is made up of the parallelly aligned one-dimensional supramolecular assembly illustrated in (b). The H₂O molecules are omitted for the sake of clarity. H gray, C pale blue, N blue, O red, Cl green, Au yellow.

FIGS. 5I-5L. Single-crystal superstructure of the adduct formed between CB[6] and HAuCl_{2.28}Br_{1.72}. (FIGS. 5I and 5K) Ball-and-stick representations showing the different views of how every CB[6] interacts with four [Au Cl_{2.28}Br_{1.72}]⁻ through [Au—X . . . H—C] (X=Cl/Br) hydrogen bonding and ion-dipole interactions. (FIG. 5J) The supramolecular assembly of the one-dimensional nanostructure extending along the b-axis, in which adjacent CB[6] molecules are connected by two parallelly aligned [AuCl_{2.28}Br_{1.72}]⁻ anions. (FIG. 5L) Solid-state superstructure of CB[6].HAuCl_{2.28}Br_{1.72}, which is made up of the parallelly aligned one-dimensional supramolecular assembly illustrated. The H₂O molecules are omitted for the sake of clarity. H gray, C pale blue, N blue, O red, Cl green, Br brown, Au yellow.

FIGS. 6A-6D. Solid-state superstructure of the adduct formed between CB[6] and HAuBr4. (FIG. 6A) The supramolecular assembly of the two-dimensional nanostructure in the a—b plane as a result of multiple hydrogen bonding between H₂O and CB[6] molecules, as well as between two adjacent CB[6] molecules. (FIG. 6B) Solid-state superstructure of CB[6].HAuBr₄, in which two polymorphs of the [AuBr_{4]}⁻ (α-[AuBr₄]⁻ and β-[AuBr₄]⁻ anions are accommodated in the lattice between the two-dimensional supramolecular assemblies illustrated in (FIG. 6A). (FIG. 6C) Ball-and-stick representation shows that every α-[AuBr_{4]}⁻ anions interact with two CB[6] molecules through [Au—Br . . . H—C] hydrogen bonding. (FIG. 6D) The β-[AuBr₄]⁻ anions interact with five CB[6] molecules, which are disordered over two position with 50:50 occupancies. H gray, C pale blue, N blue, O red, Br brown, Au yellow.

FIGS. 6E-6H. Single-crystal superstructures of the adduct formed between CB[6] and KAuBr₄. (FIG. 6E) The supramolecular assembly of the two-dimensional nanostructure through multiple ion-dipole interactions between K⁺ and CB[6] molecules, as well as hydrogen bonding between two adjacent CB[6] molecules. (FIG. 6F) Solid-state superstructure of CB[6]KAuBr4, in which two kinds of [AuBr₄]⁻—α-[AuBr₄]⁻ and β-[AuBr₄]⁻—are accommodated in the lattice space between the two-dimensional supramolecular assembly illustrated in (FIG. 6E). (FIG. 6G) Ball-and-stick representation showing that every α-[AuBr₄]⁻ interacts with two CB[6] molecules through [Au—Br . . . H—C] hydrogen bonding, and is accompanied with disordered over two position with 50:50 occupancies. (FIG. 6H) The β-[AuBr₄]⁻ interacts with five CB[6] molecules through [Au—Br . . . H—C] hydrogen bonding and ion-dipole interactions. H gray, C pale blue, N blue, O red, Br brown, K purple, Au yellow.

FIGS. 7A-7F. Solid-state superstructures and binding energies between CB[6] and [AuX₄]⁻ (X=Cl/Br) anions obtained by DFT calculations. (FIGS. 7A and 7B) Capped-sticks representation illustrating the different views of how CB[6] (A) interacts with four [AuCl₄]⁻ (1-4) anions and six adjacent CB[6] molecules (B-G) in the solid-state superstructures of CB[6].HAuCl₄. (FIG. 7C) Results of DFT calculations of the binding energies between CB[6] (A) and four connected [AuCl₄]⁻ (1-4) anions in addition to six adjacent CB[6] molecules (B-G). (FIGS. 7D and 7E) Capped-sticks representation illustrating different views of how CB[6] (A) interacts with seven [AuBr₄]⁻ (1-7) anions and four adjacent CB[6] molecules (B-E) in the solid-state superstructures of CB[6].HAuBr₄. (FIG. 7F) Results of DFT calculations of the binding energies between CB[6] (A) and seven connected [AuBr₄]⁻ anions in addition to four adjacent CB[6] molecules (B-E). A-X (X=1-7/B-G) representing two interacting molecules defined in FIGS. 7A-7F. H gray, C pale blue, N blue, O red, Cl green, Br brown, Au yellow.

FIGS. 8A-8B. (FIG. 8A) Schematic illustration of the recovery of gold from a gold-bearing material. (FIG. 8B) Gold recovery flow diagram based on the co-precipitation of CB[6].HAuCl₄. Dashed boxes indicate the flow direction of the gold recovery.

FIGS. 9A-9D. Fourier-transform infrared (FTIR) spectra for the co-precipitates of (FIG. 9A) CB[6].HAuCl₄, (FIG. 9B) CB[6].KAuCl₄, (FIG. 9C) CB[6].HAuBr₄, (FIG. 9D) CB[6]KAuBr₄, compared with the spectra of their corresponding components.

FIGS. 10A-10D. Powder X-ray diffraction patterns of the co-precipitates (FIG. 10A) CB[6]HAuCl₄, (FIG. 10B) CB[6]KAuCl₄, (FIG. 10C) CB[6].HAuBr₄, (FIG. 10D) CB[6]KAuBr₄, compared with simulated patterns derived from their X-ray crystallographic data.

FIG. 11. The photographic images of the Ostwald ripening process for the four co-precipitates CB[6]MAuX₄ (M=H/K, X=Cl/Br). It reveals that the co-precipitates of CB[6]MAuCl₄ change from co-precipitates to big crystals, respectively, while the co-precipitates of CB[6]MAuBr₄ do not exhibit much change at all.

FIGS. 12A-12B. Powder X-ray diffraction patterns for the ripe crystal of (FIG. 12A) CB[6].HAuCl₄, (FIG. 12B) CB[6] KAuCl₄, obtained by the co-precipitation of them after standing 3 days, compared with simulated patterns derived from their X-ray crystallographic data. The results reveal that there are no crystalline transformations during the Ostwald ripening process.

FIG. 13. Thermogravimetric analysis (TGA) of the co-precipitates of the four adducts CB[6].HAuCl₄, CB[6].KAuCl₄, CB[6].HAuBr₄ and CB[6].KAuBr₄.

FIG. 14. Calibration graphs for ICP-OES Au elemental analysis recorded over a range of concentrations from 0.78 to 50 ppm in 2% HCl and HNO₃ aqueous solutions, R²=0.9999.

### DETAILED DESCRIPTION OF THE INVENTION

Disclosed herein are high-efficiency gold recovery methods utilizing cucurbiturial. The composition and methods described herein provide an environmentally benign, highly efficient, and thoroughly selective processes for gold recovery. As further described herein, contacting a macrocycle with gold halide anions creates adducts where the gold halide anions are reversibly bound to the outer surface of the macrocycle by non-covalent interactions, allowing of the efficient production of precipitates that may be separated from their gold bearing source material.

The Examples demonstrate the trapping of metal halide ions, such as [AuCl₄]⁻ and [AuBr₄]⁻ anions, as their acids and alkali salts with a cucurbiturial macrocycle, such as CB[6], facilitated by the multiple weak [Au—X . . . H—C](X=Cl/Br) hydrogen bonding and [Au—X . . . C=O] (X=Cl/Br) ion-dipole interactions. After optimization of different experimental conditions, including with respect to the relative concentrations of CB[6] , MAuX₄ (M=H/K, X=Cl/Br) salts and acids (HCl), a gold recovery efficiency of 99.2% was achieved based on the co-precipitation of CB[6] and HAuCl₄. Additionally, a laboratory-scale gold recovery process was established based on the highly efficient co-precipitation of CB[6].HAuCl₄ adduct.

An “adduct”is a new chemical species AB, each molecular entity of which is formed by direct combination of two separate molecular entities A and B in such a way that there is change in connectivity, but no loss, of atoms within the moieties A and B. Stoichiometries other than 1:1 are also possible, such as 2:1, 3:1, 4:1 and so forth.

The adduct is formed from a metal halide anion non-covalently bound to the outer surface of a macrocycle. Macrocycles are a cyclic macromolecular or a macromolecular cyclic portion of a macromolecule. A molecule of high relative molecular mass, the structure of which essentially comprises the multiple repetition of units derived, actually or conceptually, from molecules of low relative molecular mass.

Suitably the macrocycle is a cucurbituril. A “cucurbituril”is macrocyclic molecule made of glycoluril (═C₄H₂N₄O₂═) monomers linked by methylene bridges (—CH₂—). Cucurbituril may generically be referred to as cucurbit[n]uril or CB[n] wherein n is the number of glycoluril units. An exemplary method of preparing CB[n] is shown in Scheme 1.

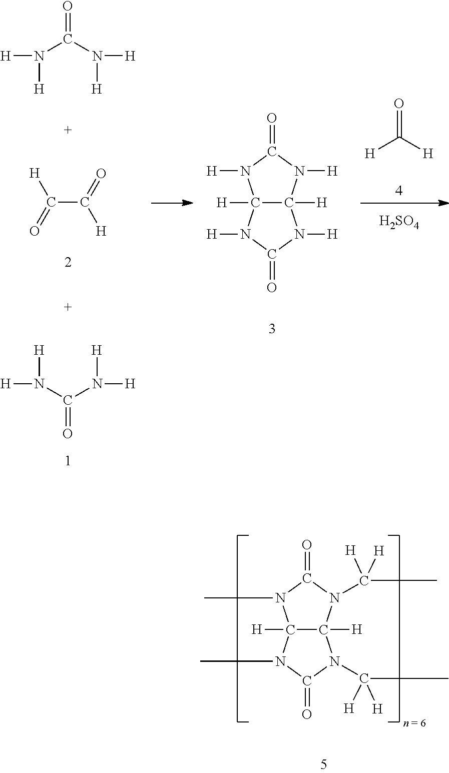

Cucurbiturils are amidals and synthesized from urea 1 and a dialdehyde (e.g., glyoxal 2) via a nucleophilic addition to give the intermediate glycoluril 3. This intermediate is condensed with formaldehyde to give hexamer cucurbit[6]uril above 110° C. Ordinarily, multifunctional monomers such as 3 would undergo a step-growth polymerization that would give a distribution of products, but due to favorable strain and an abundance of hydrogen bonding, the hexamer 5 is the only reaction product isolated after precipitation.

Other cucurbiturils may also be prepared with differing numbers of glycoluril units. Decreasing the temperature of the reaction to allows access to other sizes of cucurbiturils, including CB[5], CB[7], CB [8], and CB [10]. CB[6] may still be the major product with the other ring sizes formed in smaller yields. The isolation of sizes other than CB[6] requires fractional crystallization and dissolution.

The metal halide anion comprises a noble metal. In some embodiments, the metal halide anion is a square-planar anion such as [MX₄]⁻ where M is a noble metal, such as Au, and X is a halide. The metal halide anion may comprise other noble metals such as Pt or Pd. Each halide may be the same, such as for [AuCl₄]⁻ or [AuBr₄]⁻. In other embodiments, the metal halide anion may comprise two or more different halides. In some embodiments, the metal halide anion is provided with a counter anion such as H⁺ or metal cation, such as an alkali cation.

Superstructures and crystalline compositions may be formed from adducts described herein. A “crystalline composition” is a material whose constituents are arranged in a highly ordered microscopic structure, forming a crystal lattice. A “superstructure” is a material having additional structure superimposed upon a given crystalline material, supramolecular assembly, or other well-defined substructure.

A “supramolecular assembly” is a well-defined complex of molecules held together by noncovalent bonds. Suitably, supramolecular assemblies may have well defined order in one, two, or three dimensions. The supramolecular assemblies described herein between the gold halide anion and the outer surface of the macrocycle may include hydrogen bonds and/or ion-dipole interactions, such as [Au—X . . . H—C] and [Au—X . . . C═O], respectively.

In some embodiments, the well-defined substructure may be a supramolecular polymer. “Supramolecular polymers” are polymeric arrays of monomer units, held together by reversible and directional non-covalent interactions, such as hydrogen bonds. The resulting materials therefore maintain their polymeric properties in solution. The directions and strengths of the interactions are tuned so that the array of molecules behaves as a polymer. The high reversibility of the non-covalent bonds ensures that supramolecular polymers are always formed under conditions of thermodynamic equilibrium. The lengths of the chains are directly related to the strength of the non-covalent bond, the concentration of the monomer, and the temperature.

The compositions described herein may be used for the isolation and recovery of gold from gold-bearing materials. A “gold-bearing material” is material comprised of gold atoms, regardless of oxidation state. Exemplary gold-bearing materials include, without limitation, ores, metal mixtures, or post-consumer products.

The term “metal mixture”refers to two or more elements from Groups IA, IIA, IB to the lanthanide series and actinide series of the periodic table. An example of a metal mixture is Au and Pt.

The term “post-consumer product” refers to any man-made product for consumption, bartering, exchange or trade. Examples of “post-consumer product” include a jewelry item, an electronics item, precious metal products, and coins, among others.

The term “jewelry item” includes any aesthetic item that includes as one component a precious metal. Examples of a jewelry item include a ring, a bracelet and a necklace, among others.

The term “electronics item” refers to a product that includes at least one circuit for conducting electron flow. Examples of an electronics item include a computer, a monitor, a power supply, an amplifier, and a preamplifier, a digital to analog converter, an analog to digital converter, and a phone, among others.

The term “precious metal product” includes a partially purified form or a purified form of a noble metal, such as gold, platinum, palladium and silver. Examples of a precious metal include a powder, ingot, or bar of gold, silver, platinum, among others. As used herein, “partially-purified form” refers to a form having from about 10% to about 75% of the pure form of a noble metal. As used herein, “purified form” refers to a form having greater than about 75% of the pure form of a noble metal.

The term “coin” refers to any pressed object composed of a pure metal, mixed metal or metal alloy that can be used as a currency, a collectable, among other uses. As used herein, “pure metal” refers to a single metal of at least 95% or greater purity. As used herein “mixed metal” refers to two or more metals. As used herein “metal alloy” refers to a mixture or solid solution of a metal with at least one other element.

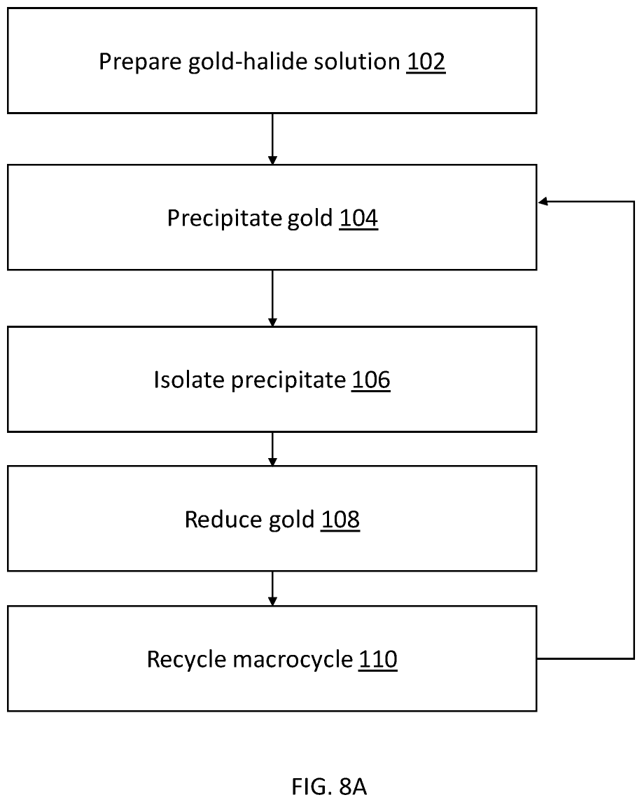

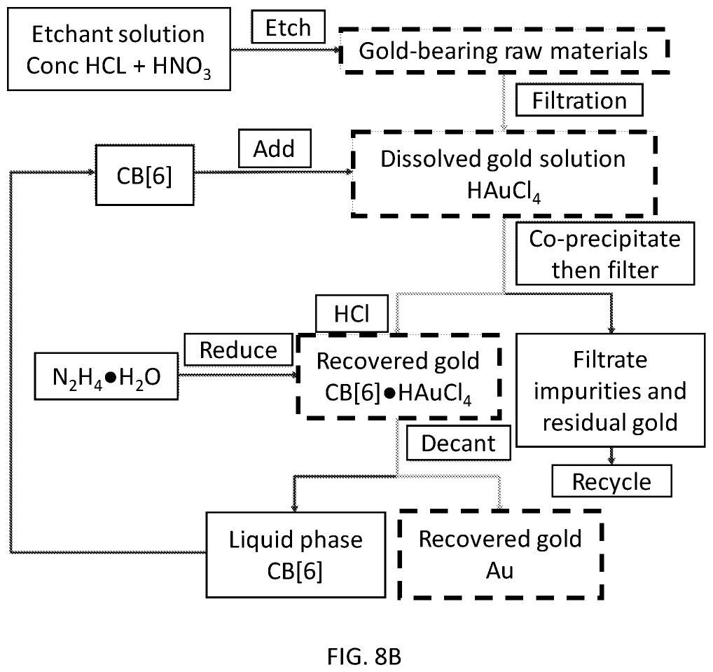

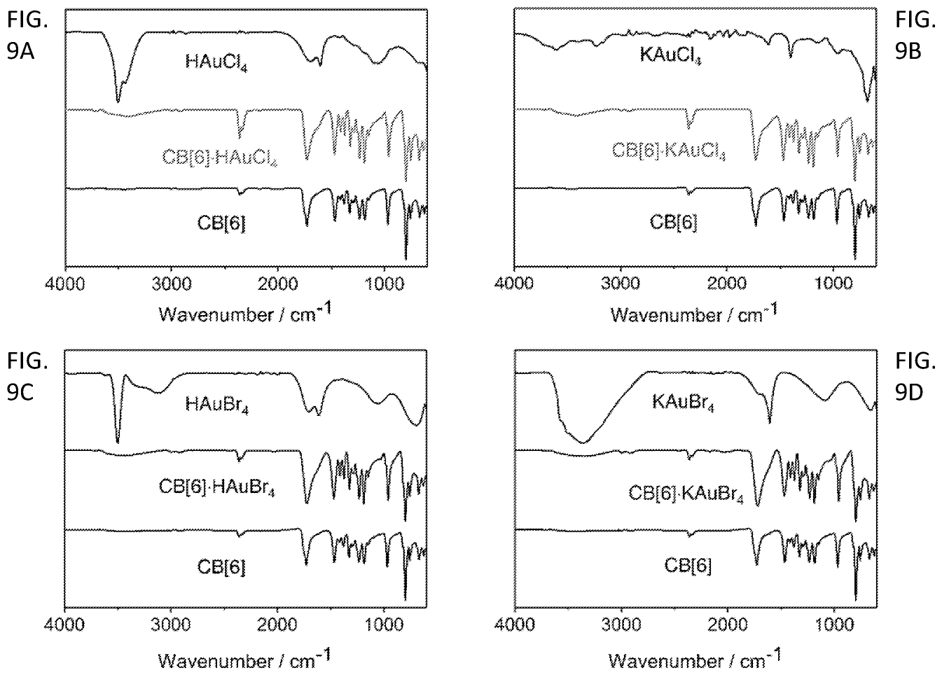

A method for isolating and recovering gold from gold-bearing materials was developed based upon the selective co-precipitation of metal halide anions non-covalently bound to the outer surface of macrocycles. Referring to FIG. 8A, a gold-bearing material is combined with a hydrogen halide (“HX”) and, optionally, an acid to form a gold halide solution 102. Hydrogen halide can be any compound having the formula HX, wherein X is a halogen such as chlorine or bromine. The optional acid can include any strong acid, such as any of the foregoing hydrogen halides, or additionally HNO₃, H₂SO₄, among others. The pH of the gold halide solution may be less than 4.0 or, in some embodiments, less than 3.0, or less than 2.0. The gold of the gold-bearing material reacts with the hydrogen halide to form the product HAuX₄.

The macrocycle may be added to the gold halide solution to form a precipitate 104. Suitably the macrocycle is a cucurbituril, such as CB[6]. The precipitate may comprises any of the adducts, superstructure, or crystalline materials described herein. In some embodiments, the precipitate [AuX₄]⁻ is bound to the outer surface of CB[6].

The precipitate is isolated from the metal halide salutation 106. Any means of isolation can be used to obtain precipitate, include filtration, centrifugation, and other separation methods known in the art.

In some embodiments, not all of gold-bearing material can be dissolved in gold halide solution. As a result, some solid remnants of gold-bearing material (whether or not including gold) can persist. In such aspects, it may be desirable to include a filtration step to remove the solid remnants prior to subsequent processing. The resultant filtrate may be processed as described above to obtain the isolated gold.

The precipitate can be treated with a reducing agent to produce elemental gold (Au(0)) 108. Examples of reducing agents include, but are not limited to, N₂H₄, NaBH₄, Na₂S₂O₅, and H₂C₂O₄, among others. The elemental gold can be readily isolated as a precipitate and the macrocycle can be harvested in the liquid phase and recycled for reuse to precipitate additional gold 110.

An exemplary aspect of the process outlined generally in FIG. 8A is depicted in FIG. 8B. The method for isolating and recovering gold from gold-bearing materials has several applications. In one aspect, the method can be applied to isolating gold from gold-bearing material, wherein the gold-bearing material is selected from an ore, a metal mixture, or a post-consumer product. The foregoing examples of isolating gold from gold-bearing materials are not limited to the foregoing materials. The specific etching and leaching process for dissolving gold from gold-bearing materials results in formation of a specific gold-halide compound that can be recovered in the form of a complex with the macrocycle, thereby rendering the method suitable for recovering gold from each of these particular applications as well as other gold-bearing materials.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus>10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

(1) Nnorom, I. C.; Osibanjo, O.; Nnorom, S. O. Achieving Resource Conservation in Electronic Waste Management: A Review of Options Available to Developing Countries. J. Applied Sci. 2007, 7, 2918-2933.
(2) Ilankoon, I.; Ghorbani, Y; Chong, M. N.; Herath, G.; Moyo, T.; Petersen, J. E-Waste in the International Context—A Review of Trade Flows, Regulations, Hazards, Waste Management Strategies and Technologies for Value Recovery. Waste Manage. 2018, 82, 258-275.
(3) Diaz-Barriga-Fernandez, A. D.; Santibanez-Aguilar, J. E.; Radwan, N.; Napoles-Rivera, F.; El-Halwagi, M. M.; Ponce-Ortega, J. M. Strategic Planning for Managing Municipal Solid Wastes with Consideration of Multiple Stakeholders. ACS Sustainable Chem. Eng. 2017, 5, 10744-10762.
(4) (a) Scanlon, M. D.; Smirnov, E.; Stockmann, T. J.; Peljo, P. Gold Nanofilms at Liquid-Liquid Interfaces: An Emerging Platform for Redox Electrocatalysis, Nanoplasmonic Sensors, and Electrovariable Optics. Chem. Rev. 2018, 118, 3722-3751. (b) Zhu, B.; Gong, S.; Cheng, W. Softening Gold for Elastronics. Chem. Soc. Rev. 2019, 48, 1668-1711.
(5) Hagelüken, C.; Corti, C. W. Recycling of Gold from Electronics: Cost-Effective Use through ‘Design for Recycling’. Gold Bull. 2010, 43, 209-220.
(6) Norgate, T.; Hague, N. Using Life Cycle Assessment to Evaluate Some Environmental Impacts of Gold Production. J. Clean. Prod. 2012, 29-30, 53-63.
(7) Veiga, M. M.; Nunes, D.; Klein, B.; Shandro, J. A.; Velasquez, P. C.; Sousa, R. N. Mill Leaching: A Viable Substitute for Mercury Amalgamation in the Artisanal Gold Mining Sector? J. Clean. Prod. 2009, 17, 1373-1381.
(8) Jaszczak, E.; Polkowska, Z.; Narkowicz, S.; Namiesnik, J. Cyanides in the Environment-Analysis-Problems and Challenges. Environ. Sci. Pollut. Res. Int. 2017, 24, 15929-15948.
(9) (a) Zhang, Y.-H.; Zhang, Y.-M.; Chen, Y.; Yang, Y.; Liu, Y. Phenanthroline Bridged Bis((β-cyclodextrin)s/Adamantane-Carboxylic Acid Supramolecular Complex as an Efficient Fluorescence Sensor to Zn²⁺. Org. Chem. Front. 2014, 1, 355-360. (b) Prochowicz, D.; Kornowicz, A.; Lewinski, J. Interactions of Native Cyclodextrins with Metal Ions and Inorganic Nanoparticles: Fertile Landscape for Chemistry and Materials Science. Chem. Rev. 2017, 117, 13461-13501.
(10) (a) Pedersen, C. J. Cyclic Polyethers and Their Complexes with Metal Salts. J. Am. Chem. Soc. 1967, 89, 2495-2496. (b) Pedersen, C. J. Cyclic Polyethers and Their Complexes with Metal Salts. J. Am. Chem. Soc. 1967, 89, 7017-7036.
(11) Homden, D. M.; Redshaw, C. The Use of Calixarenes in Metal-Based Catalysis. Chem. Rev. 2008, 108, 5086-5130.
(12) (a) Chen, L.; Cai, Y; Feng, W.; Yuan, L. Pillararenes as Macrocyclic Hosts: A Rising Star in Metal Ion Separation. Chem. Commun. 2019, 55, 7883-7898. (b) Dai, D.; Li, Z.; Yang, J.; Wang, C.; Wu, J. R.; Wang, Y; Zhang, D.; Yang, Y. W. Supramolecular Assembly-Induced Emission Enhancement for Efficient Mercury(II) Detection and Removal. J. Am. Chem. Soc. 2019, 141, 4756-4763.
(13) He, Q.; Williams, N. J.; Oh, J. H.; Lynch, V. M.; Kim, S. K.; Moyer, B. A.; Sessler, J. L. Selective Solid-Liquid and Liquid-Liquid Extraction of Lithium Chloride Using Strapped Calix[4]pyrroles. Angew. Chem. Int. Ed. 2018, 57, 11924-11928.
(14) Atwood, J. L.; Orr, G. W.; Hamada, F.; Vincent, R. L.; Bott, S. G.; Robinson, K. D. Calixarenes as Second-Sphere Ligands for Transition Metal Ions. Synthesis and Crystal Structure of [(H₂O)₅Ni(NC₅H₅)]₂(Na)[calix[4]arene sulfonate].3.5 H₂O and [(H₂O)₄Cu(NC₅H₅)₂]—(H₃O)₃[calix [4]arene sulfonate].10 H₂O. J. Inclusion Phenom. Mol. Recognit. Chem. 1992, 14, 37-46.
(15) Alston, D. R.; Slawin, A. M. Z.; Stoddart, J. F.; Williams, D. J. Cyclodextrins as Second Sphere Ligands for Transition Metal Complexes—The X-Ray Crystal Structure of [Rh(cod)(NH₃)₂.α-cyclodextrin][PF₆].6H₂O. Angew. Chem. Int. Ed. Engl. 1985, 24, 786-787.
(16) Hua, B.; Shao, L.; Zhang, Z.; Liu, J.; Huang, F. Cooperative Silver Ion-Pair Recognition by Peralkylated Pillar[5]arenes. J. Am. Chem. Soc. 2019, 141, 15008-15012.
(17) Ohashi, M.; Konkol, M.; Del Rosal, I.; Poteau, R.; Maron, L.; Okuda, J. Rare-Earth Metal Alkyl and Hydride Complexes Stabilized by a Cyclen-Derived [NNNN] Macrocyclic Ancillary Ligand. J. Am. Chem. Soc. 2008, 130, 6920-6921.
(18) Colquhoun, H. M.; Stoddart, J. F.; Williams, D. J. The Binding of Neutral Platinum Complexes by Crown Ethers. X-Ray Crystal Structures of [trans-PtCl₂(PMe₃)NH₃.dibenzo-18-crown-6] and [{trans-PtCl₂(PMe₃)NH₃}₂.18-crown-6]. J. Chem. Soc., Chem. Commun. 1981, 0, 847-849.
(19) Kiegiel, K.; Steczek, L.; Zakrzewska-Trznadel, G. Application of Calixarenes as Macrocyclic Ligands for Uranium(VI): A Review. J. Chem. 2013, 2013, 1-16.
(20) (a) Liu, Z.; Frasconi, M.; Lei, J.; Brown, Z. J.; Zhu, Z.; Cao, D.; Iehl, J.; Liu, G.; Fahrenbach, A. C.; Botros, Y. Y.; Farha, O. K.; Hupp, J. T.; Mirkin, C. A.; Stoddart, J. F. Selective Isolation of Gold Facilitated by Second-Sphere Coordination with a-Cyclodextrin. Nat. Commun. 2013, 4, 1855. (b) Liu, Z.; Samanta, A.; Lei, J.; Sun, J.; Wang, Y.; Stoddart, J. F. Cation-Dependent Gold Recovery with α-Cyclodextrin Facilitated by Second-Sphere Coordination. J. Am. Chem. Soc. 2016, 138, 11643-11653.
(21) Chen, L. X.; Liu, M.; Zhang, Y. Q.; Zhu, Q. J.; Liu, J. X.; Zhu, B. X.; Tao, Z. Outer Surface Interactions to Drive Cucurbit[8]uril-Based Supramolecular Frameworks: Possible Application in Gold Recovery. Chem. Commun. 2019, 55, 14271-14274.
(22) (a) Freeman, W. A.; Mock, W. L.; Shih, N. Y Cucurbituril. J. Am. Chem. Soc. 1981, 103, 7367-7368. (b) Kim, J.; Jung, I.-S.; Kim, S.-Y.; Lee, E.; Kang, J.-K.; Sakamoto, S.; Yamaguchi, K.; Kim, K. New Cucurbituril Homologues: Syntheses, Isolation, Characterization, and X-ray Crystal Structures of Cucurbit[n]uril (n=5,7, and 8). J. Am. Chem. Soc. 2000, 122, 540-541. (c) Lagona, J.; Mukhopadhyay, P.; Chakrabarti, S.; Isaacs, L. The cucurbit[n]uril family. Angew. Chem. Int. Ed. 2005, 44, 4844-4870. (d) Barrow, S. J.; Kasera, S.; Rowland, M. J.; del Barrio, J.; Scherman, O. A. Cucurbituril-Based Molecular Recognition. Chem. Rev. 2015, 115, 12320-12406.
(23) (a) Jeon, Y-M.; Kim, J.; Whang, D.; Kim, K. Molecular Container Assembly Capable of Controlling Binding and Release of Its Guest Molecules: Reversible Encapsulation of Organic Molecules in Sodium Ion Complexed Cucurbituril. J. Am. Chem. Soc. 1996, 118, 9790-9791. (b) Heo, J.; Kim, S.-Y; Whang, D.; Kim, K. Shape-Induced, Hexagonal, Open Frameworks: Rubidium Ion Complexed Cucurbituril. Angew. Chem. Int. Ed. 1999, 38, 641-643. (c) Thuéry, P. Uranyl-Alkali Metal Ion Heterometallic Complexes with Cucurbit[6]uril and a Sulfonated Catechol. Cryst. Growth Des. 2011, 11, 3282-3294.
(24) Gerasko, O. A.; Mainicheva, E. A.; Naumova, M. I.; Neumaier, M.; Kappes, M. M.; Lebedkin, S.; Fenske, D.; Fedin, V. P. Sandwich-Type Tetranuclear Lanthanide Complexes with Cucurbit[6]uril: From Molecular Compounds to Coordination Polymers. Inorg. Chem. 2008, 47, 8869-8880.
(25) (a) Samsonenko, D. G.; Sokolov, M. N.; Gerasko, O. A.; Virovets, A. V.; Lipkowski, J.; Fenske, D.; Fedin, V. P. Syntheses and Crystal Structures of SmIII and ThIV complexes with Macrocyclic Cavitand Cucurbituril. Russ. Chem. Bull. 2003, 52, 2132-2139. (b) Thuery, P. Uranyl-Lanthanide Heterometallic Complexes with Cucurbit[6]uril and Perrhenate Ligands. Inorg. Chem. 2009, 48, 825-827.
(26) Ghale, G.; Nau, W. M. Dynamically Analyte-Responsive Macrocyclic Host-Fluorophore Systems. Acc. Chem. Res. 2014, 47, 2150-2159.
(27) Zhao, N.; Lloyd, G. O.; Scherman, O. A. Monofunctionalised Cucurbit[6]uril Synthesis Using Imidazolium Host-guest Complexation. Chem. Commun. 2012, 48, 3070-3072.
(28) (a) Lim, S.; Kim, H.; Selvapalam, N.; Kim, K. J.; Cho, S. J.; Seo, G.; Kim, K. Cucurbit[6]uril: Organic Molecular Porous Material with Permanent Porosity, Exceptional Stability, and Acetylene Sorption Properties. Angew. Chem. Int. Ed. 2008, 47, 3352-3355. (b) Kim, H.; Kim, Y; Yoon, M.; Lim, S.; Park, S. M.; Seo, G.; Kim, K. Highly Selective Carbon Dioxide Sorption in an Organic Molecular Porous Material. J. Am. Chem. Soc. 2010, 132, 12200-12202.
(29) (a) Lee, H. K.; Park, K. M.; Jeon, Y J.; Kim, D.; Oh, D. H.; Kim, H. S.; Park, C. K.; Kim, K. Vesicle Formed by Amphiphilc Cucurbit[6]uril: Versatile, Noncovalent Modification of the Vesicle Surface, and Multivalent Binding of Sugar-Decorated Vesicles to Lectin. J. Am. Chem. Soc. 2005, 127, 5006-5007. (b) Wu, X.; Zhang, Y.-M.; Liu, Y Nanosupramolecular Assembly of Amphiphilic Guest Mediated by Cucurbituril for Doxorubicin Delivery. RSC Adv. 2016, 6, 99729-99734.
(30) (a) Ke, C.; Smaldone, R. A.; Kikuchi, T.; Li, H.; Davis, A. P.; Stoddart, J. F. Quantitative Emergence of Hetero[4]rotaxanes by Template-Directed Click Chemistry. Angew. Chem. Int. Ed. 2013, 52, 381-387. (b) Ren, H.; Huang, Z.; Yang, H.; Xu, H.; Zhang, X. Controlling the Reactivity of the Se—Se Bond by the Supramolecular Chemistry of Cucurbituril. Chemphyschem 2015, 16, 523-257. (c) Finbloom, J. A.; Han, K.; Slack, C. C.; Furst, A. L.; Francis, M. B. Cucurbit[6]uril-Promoted Click Chemistry for Protein Modification. J. Am. Chem. Soc. 2017, 139, 9691-9697.
(31) (a) Kim, K.; Jeon, W. S.; Kang, J. K.; Lee, J. W.; Jon, S. Y; Kim, T.; Kim, K. A Pseudorotaxane on Gold: Formation of Self-Assembled Monolayers, Reversible Dethreading and Rethreading of the Ring, and Ion-Gating Behavior. Angew. Chem. Int. Ed. 2003, 42, 2293-2296. (b) Angelos, S.; Khashab, N. M.; Yang, Y. W.; Trabolsi, A.; Khatib, H. A.; Stoddart, J. F.; Zink, J. I. pH Clock-Operated Mechanized Nanoparticles. J. Am. Chem. Soc. 2009, 131, 12912-12914. (c) Park, K. M.; Yang, J. A.; Jung, H.; Yeom, J.; Park, J. S.; Park, K. H.; Hoffman, A. S.; Hahn, S. K.; Kim, K. In Situ Supramolecular Assembly and Modular Modification of Hyaluronic Acid Hydrogels for 3D Cellular Engineering. ACS Nano. 2012, 6, 2960-2968.
(32) (a) Zhang, F.; Yajima, T.; Li, Y. Z.; Xu, G. Z.; Chen, H. L.; Liu, Q. T.; Yamauchi, O. Iodine-Assisted Assembly of Helical Coordination Polymers of Cucurbituril and Asymmetric Copper(II) Complexes. Angew. Chem. Int. Ed. 2005, 44, 3402-3407. (b) Lin, R.-G.; Long, L.-S.; Huang, R.-B.; Zheng, L.-S. Directing Role of Hydrophobic—Hydrophobic and Hydrophilic—Hydrophilic Interactions in the Self-Assembly of Calixarenes/Cucurbiturils-Based Architectures. Cryst. Growth Des. 2008, 8, 791-794.
(33) (a) Ni, X. L.; Xiao, X.; Cong, H.; Liang, L. L.; Cheng, K.; Cheng, X. J.; Ji, N. N.; Zhu, Q. J.; Xue, S. F.; Tao, Z. Cucurbit[n]uril-Based Coordination Chemistry: from Simple Coordination Complexes to Novel Poly-Dimensional Coordination Polymers. Chem. Soc. Rev. 2013, 42, 948-508. (b) Wu, H.; Chen, Y.; Dai, X.; Li, P.; Stoddart, J. F.; Liu, Y. In Situ Photoconversion of Multicolor Luminescence and Pure White Light Emission Based on Carbon Dot-Supported Supramolecular Assembly. J. Am. Chem. Soc. 2019, 141, 6583-6591.
(34) (a) Johnson, N. J.; Korinek, A.; Dong, C.; van Veggel, F. C. Self-Focusing by Ostwald Ripening: A Strategy for Layer-by-Layer Epitaxial Growth on Upconverting Nanocrystals. J. Am. Chem. Soc. 2012, 134, 11068-11071. (b) Ouyang, R.; Liu, J. X.; Li, W. X. Atomistic Theory of Ostwald Ripening and Disintegration of Supported Metal Particles under Reaction Conditions. J. Am. Chem. Soc. 2013, 135, 1760-1771. (c) Liu, L.; Chen, Z.; Wang, J.; Zhang, D.; Zhu, Y.; Ling, S.; Huang, K. W.; Belmabkhout, Y; Adil, K.; Zhang, Y; Slater, B.; Eddaoudi, M.; Han, Y. Imaging Defects and Their Evolution in a Metal-Organic Framework at Sub-Unit-Cell Resolution. Nat. Chem. 2019, 11, 622-628.

### EXAMPLES

Herein, we report a highly efficient gold recovery protocol on the basis of the instantaneous assembly between cucurbitu[6]ril (CB[6]) and [AuX₄]⁻ (X=Cl/Br) anions. Upon mixing CB[6] and the gold-bearing salts such as MAuX₄ (M=H/K, X=Cl/Br) in aqueous solutions, yellow or brown precipitates form immediately, benefiting from multiple weak [Au—X . . . H—C] (X=Cl/Br) hydrogen bonding and [Au—X . . . C=O] (X=Cl/Br) ion-dipole interactions between CB[6] and [AuX₄]⁻ anions. The combination of CB[6] and HAuCl₄ affords the highest yield (99.2%) under optimized conditions. In the crystal superstructures of all the four adducts, [AuCl₄]⁻ anions and CB[6] molecules adopt an alternating arrangement to form doubly connected supramolecular polymers, while [AuBr₄]⁻ anions are accommodated in the lattice between two-dimensional layered nanostructures composed of CB[6] molecules. DFT Calculations have revealed that the binding energy (34.8 kcal mol⁻¹) between CB[6] and [AuCl₄]⁻ anion is higher than that (11.3-31.3 kcal mol⁻¹) of CB[6] and [AuBr₄]⁻ anion, which leads to the better crystallinity as well as the higher yield of CB[6].MAuCl₄ (M=H K) co-precipitates. Additionally, a laboratory-scale gold recovery process was established based on the highly efficient co-precipitation of CB[6].HAuCl₄, which exhibits an application potential of current co-precipitation strategy for practical gold recovery. The use of CB[6] as a gold extractant provides for new opportunities to develop more efficient and environmentally friendly processes for the industrial production of gold.

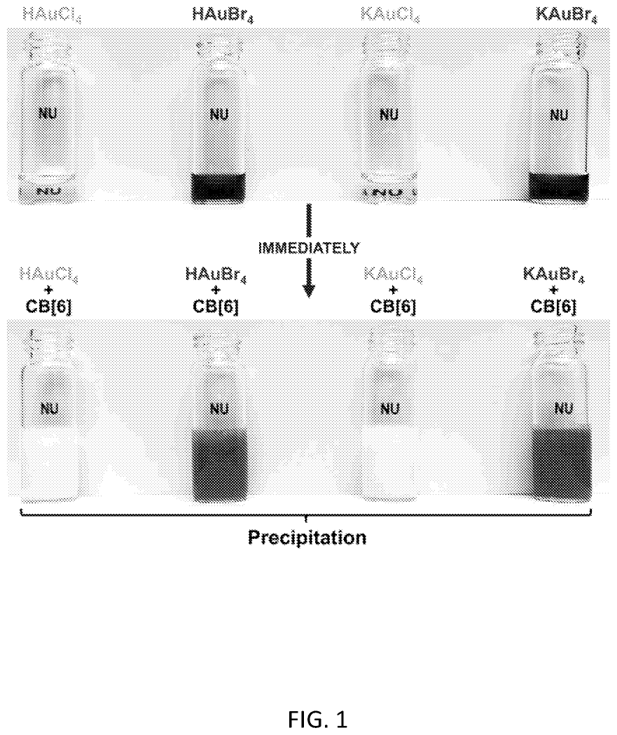

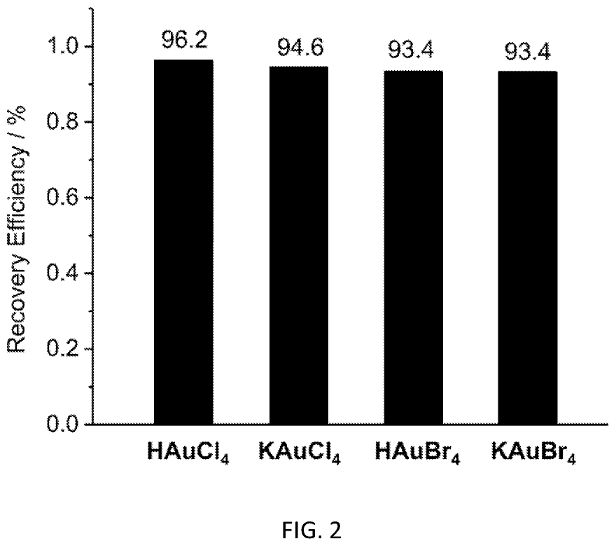

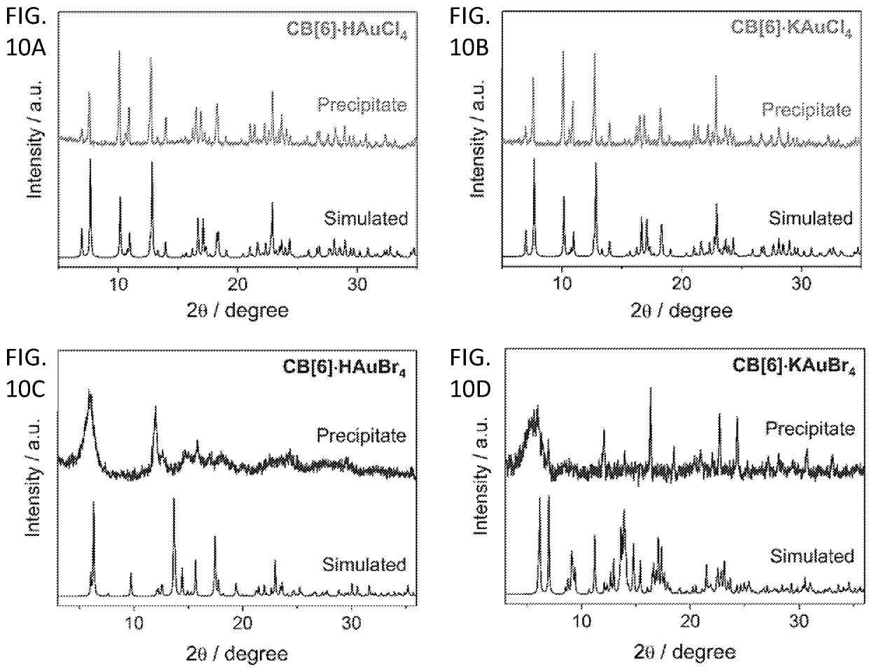

Upon mixing any particular aqueous solution of KAuX₄ (X=Cl/Br, 20.0 mM, 0.6 mL) with an aqueous solution of CB[6] (8.0 mM, 1.5 mL) containing HCl (3.0 M) or HBr (3.5 M) at room temperature, yellow or brown co-precipitates form immediately (FIG. 1). In order to explore the nature of formation of these co-precipitates, HAuX₄ (X=Cl/Br) were employed as control compounds. When CB[6] (8.0 mM, 1.5 mL) in an aqueous HCl (3.0 M) or HBr (3.5 M) solution was added to either aqueous solutions of HAuCl₄ or HAuBr₄ (20.0 mM, 0.6 mL), respectively, yellow or brown co-precipitates (FIG. 1) also formed immediately. These observations established the fact that the [AuX₄]⁻ (X=Cl/Br) anions can form co-precipitates with CB[6] either with or without K⁺ ions, indicating that gold halide anions [AuX₄]⁻ play a role in the formation of the co-precipitates. Centrifugal filtration and air drying of the co-precipitates permit the isolation of the four complexes—which are identified by the descriptors CB[6].HAuCl₄, CB[6].KAuCl₄, CB[6].KAuBr₄, CB[6].KAuBr₄—in bulk as yellow or brown powder. The Fourier transform infrared (FTIR) spectra of the four powders provide (FIGS. 9A- 9D) qualitative evidence for the formation of the adducts. In order to quantify gold recovery efficiencies based on these co-precipitates, all four filtrates obtained by filtration, were diluted and subjected to inductively coupled plasma optical emission spectroscopy (ICP-OES) analysis to determine the concentrations of [AuX₄]⁻ anions remaining in the filtrates. On the basis of the initial and residual concentrations of the [AuX₄]⁻ anions in the aqueous solutions, the yields of the precipitated [AuX₄]⁻ anions can be calculated. The results reveal (FIG. 2 and Table 1)) that gold recovery yields for all the combinations between CB[6] and MAuX₄ (M=H/K, X=Cl/Br) are over 90.0% at the concentrations of 5.7 mM, which is higher than that (78%) for the co-precipitate formed between α-CD and KAuBr₄.^{[20a]} HAuCl₄ affords the higher gold recovery yield of 96.2% compared with that (94.6%) of KAuCl₄. By contrast, the HAuBr₄ and KAuBr₄ give equal and lowest gold recovery yields of 93.4%. These observations demonstrate that the co-precipitation yields depend on the nature of the [AuX₄]⁻ anions, while the metal counter cations have a relatively weak influence on the co-precipitate process.

TABLE 1
The Gold Recovery Efficiencies for the Four Co-Precipitates
of CB[6]•MAuX₄ (M = H/K, X = Cl/Br)

| | Au | Au | Au | Avg. | Au in | Total | Recovery |
|---|---|---|---|---|---|---|---|
| | 267.595/ppm | 208.209/ppm | 242.795/ppm | Au/ppm | Filtrate/mg | Au/mg | Efficiency |
| CB[6]•HAuCl₄ | 0.970 | 1.002 | 1.046 | 1.006 | 0.023 | 0.591 | 0.962 |
| CB[6]•KAuCl₄ | 1.299 | 1.320 | 1.651 | 1.423 | 0.032 | 0.591 | 0.946 |
| CB[6]•HAuBr₄ | 1.412 | 1.403 | 1.630 | 1.482 | 0.039 | 0.591 | 0.934 |
| CB[6]•KAuBr₄ | 1.410 | 1.419 | 1.633 | 1.487 | 0.039 | 0.591 | 0.934 |

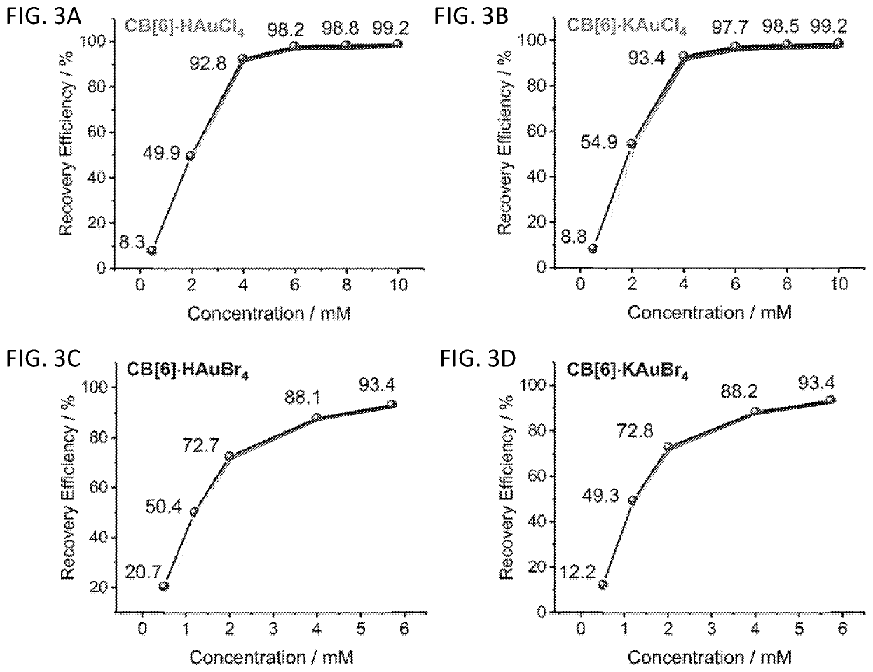

Given the potential applications of CB[6] as an eco-friendly gold extractant, the yields of the CB[6]MAuX₄ (M=H/K, X=Cl/Br) co-precipitates were optimized with respect to the concentrations of CB[6] and MAuX₄. Different concentrations (0.5, 2.0, 4.0, 6.0, 8.0 and 10.0 mM) of MAuCl₄ (M=H/K) were prepared by dissolving MAuCl₄ salts in aqueous HCl (2.0 M) solutions. Upon adding equimolar amounts of an aqueous CB[6] solution, containing HCl (2.0 M), to aqueous MAuCl₄ solutions, yellow co-precipitates formed immediately. The resulting co-precipitates were filtered immediately (<5 s), and the concentrations of the [AuCl₄]⁻ anions remaining in individual filtrates were analyzed by ICP-OES elemental analysis. The results reveal (FIG. 3A and Table 2) that gold recovery efficiencies, based on the CB[6].HAuCl₄ co-precipitate, changes dramatically from 8.3 to 92.8% upon increasing the concentrations of CB[6].HAuCl₄ from 0.5 to 4.0 mM in aqueous HCl (2.0 M) solution. When the concentrations of CB[6].HAuCl₄ were increased gradually to 10.0 mM, the yield of the co-precipitates reached 99.2% in the aqueous HCl (2.0 M) solution. Gold recovery efficiencies, based on the CB[6].HAuCl₄ co-precipitate, were (FIG. 3B and Table 3) analogous to those for CB[6].HAuCl₄ at different concentrations, and, once again increased with increasing the concentration of the adduct. These observations suggest that the higher initial concentration of the CB[6] and MAuCl₄ results of higher gold recovery efficiency.

TABLE 2
The Gold Recovery Efficiency Changes with the Concentration
of CB[6]•HAuCl₄ in 2M HCl Aqueous Solutions

| Concentration of | Au | Au | Au | Avg. | Au in | Total | Recovery |
|---|---|---|---|---|---|---|---|
| CB[6]•HAuCl₄/M | 267.595/ppm | 208.209/ppm | 242.795/ppm | Au/ppm | Filtrate/mg | Au/mg | Efficiency |
| 10.0 | 0.319 | 0.308 | 0.305 | 0.310 | 0.009 | 1.182 | 0.992 |
| 8.0 | 0.377 | 0.381 | 0.372 | 0.377 | 0.014 | 1.182 | 0.988 |
| 6.0 | 0.416 | 0.411 | 0.410 | 0.413 | 0.010 | 0.591 | 0.982 |
| 4.0 | 1.147 | 1.125 | 1.144 | 1.139 | 0.043 | 0.591 | 0.928 |
| 2.0 | 3.970 | 3.925 | 3.953 | 3.949 | 0.296 | 0.591 | 0.499 |
| 0.5 | 1.812 | 1.792 | 1.815 | 1.806 | 0.072 | 0.079 | 0.083 |

TABLE 3
The Gold Recovery Efficiency Changes with the Concentration
of CB[6]•KAuCl₄ in 2M HCl Aqueous Solutions

| Concentration of | Au | Au | Au | Avg. | Au in | Total | Recovery |
|---|---|---|---|---|---|---|---|
| CB[6]•KAuCl₄/M | 267.595/ppm | 208.209/ppm | 242.795/ppm | Au/ppm | Filtrate/mg | Au/mg | Efficiency |
| 10.0 | 0.331 | 0.287 | 0.329 | 0.316 | 0.009 | 1.182 | 0.992 |
| 8.0 | 0.471 | 0.475 | 0.454 | 0.467 | 0.018 | 1.182 | 0.985 |
| 6.0 | 0.548 | 0.529 | 0.539 | 0.539 | 0.013 | 0.591 | 0.977 |
| 4.0 | 1.056 | 1.029 | 1.029 | 1.038 | 0.039 | 0.591 | 0.934 |
| 2.0 | 3.571 | 3.559 | 3.524 | 3.551 | 0.266 | 0.591 | 0.549 |
| 0.5 | 1.814 | 1.763 | 1.812 | 1.796 | 0.072 | 0.079 | 0.088 |

By contrast, when equimolar amounts of CB[6] in the aqueous HBr (2.5 M) solution were added to the different concentrations (0.5, 1.2, 2.0, 4.0, and 5.7 mM) of MAuBr₄ (M=H/K) aqueous solution, gold recovery efficiency, based on the CB[6].HAuCl₄ and CB[6].KAuBr₄ co-precipitates, increased from 20.7 to 93.4% (FIG. 3C and Table 4) and 12.2 to 93.4% (FIG. 3D and Table 5), respectively. The limited solubility of CB[6] in aqueous HBr solution, however, hampers the further increase of the initial concentration of CB[6] and MAuBr₄, limiting gold recovery efficiency, based on CB[6].MAuBr₄ co-precipitations, to 93.4%. On the basis of these profiles of gold recovery efficiencies with varying concentration of CB[6] and MAuX₄, it can be concluded that higher initial concentrations of CB[6] and MAuX₄ will lead to the higher gold recovery efficiencies. On account of the better solubility of CB[6] in the aqueous HCl solutions than in aqueous HBr solutions, the gold recovery efficiency of CB[6].MAuCl₄ co-precipitates are higher than that of CB[6].MAuBr₄, indicating the [AuCl₄]⁻ anions are the better candidate for gold recovery process.

TABLE 4
The Gold Recovery Efficiency Changes with the Concentration
of CB[6]•HAuBr₄ in 2.5M HBr Aqueous Solutions

| Concentration of | Au | Au | Au | Avg. | Au in | Total | Recovery |
|---|---|---|---|---|---|---|---|
| CB[6]•HAuBr₄/M | 267.595/ppm | 208.209/ppm | 242.795/ppm | Au/ppm | Filtrate/mg | Au/mg | Efficiency |
| 5.7 | 1.412 | 1.403 | 1.630 | 1.482 | 0.039 | 0.591 | 0.934 |
| 4.0 | 1.866 | 1.905 | 1.861 | 1.877 | 0.070 | 0.591 | 0.881 |
| 2.0 | 2.144 | 2.170 | 2.137 | 2.150 | 0.161 | 0.591 | 0.727 |
| 1.2 | 2.277 | 2.308 | 2.452 | 2.345 | 0.195 | 0.394 | 0.504 |
| 0.5 | 1.564 | 1.555 | 1.567 | 1.562 | 0.047 | 0.059 | 0.207 |

TABLE 5
The Gold Recovery Efficiency Changes with the Concentration
of CB[6]•KAuBr₄ in 2.5M HBr Aqueous Solutions

| Concentration of | Au | Au | Au | Avg. | Au in | Total | Recovery |
|---|---|---|---|---|---|---|---|
| CB[6]•KAuBr₄/M | 267.595/ppm | 208.209/ppm | 242.795/ppm | Au/ppm | Filtrate/mg | Au/mg | Efficiency |
| 5.7 | 1.410 | 1.419 | 1.633 | 1.487 | 0.039 | 0.591 | 0.934 |
| 4.0 | 1.846 | 1.865 | 1.846 | 1.852 | 0.069 | 0.591 | 0.882 |
| 2.0 | 2.138 | 2.152 | 2.132 | 2.141 | 0.161 | 0.591 | 0.728 |
| 1.2 | 2.345 | 2.373 | 2.459 | 2.392 | 0.200 | 0.394 | 0.493 |
| 0.5 | 1.718 | 1.751 | 1.718 | 1.729 | 0.052 | 0.059 | 0.122 |

Taking into account that the concentration of acid in gold-bearing solution may vary in practice, gold recovery efficiencies based on CB[6].MAuCl₄ co-precipitates has been optimized with respect to the concentration of HCl. Six aqueous solutions of MAuCl₄ (6.0 mM), corresponding to HCl concentrations of 1.0, 2.0, 4.0, 6.0, 8.0 and 10.0 M, were prepared. Upon the addition of equimolar amounts of CB[6] in aqueous solutions with corresponding concentrations of HCl, the mixtures with 1.0 and 2.0 M of HCl generated copious amounts of co-precipitates, while the mixtures with 4.0 and 6.0 M of HCl resulted in small amounts of co-precipitates. By contrast, there were no obvious co-precipitate for the mixtures with 8.0 and 10.0 M of HCl. It should be mentioned that the CB[6] could not be dissolved completely in 1.0 M HCl aqueous solution. Then, all of the co-precipitates were removed by filtration, and the concentrations of the [AuCl₄]⁻ anions remaining in the filtrates were measured by the ICP-OES analysis. Gold recovery efficiencies, based on the co-precipitates at different concentrations of HCl, were calculated according to the initial and residue concentrations of the [AuCl₄]⁻ anions in aqueous HCl solutions.

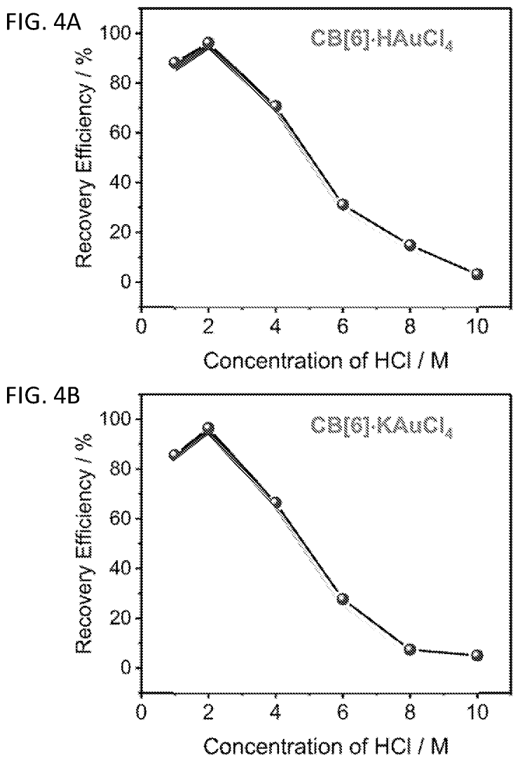

The results reveal (FIG. 4A and Table 6) that the CB[6].HAuCl₄ co-precipitates with 2.0 M HCl affords the highest gold recovery efficiency of 96.4%, while gold recovery efficiency, based on the CB[6].HAuCl₄ co-precipitate with 1.0 M HCl, is 88.4%. This observation maybe because of the lower solubility of the CB[6] in the aqueous 1.0 M HCl solution, leading to the actual concentration of CB[6] being lower than 6.0 mM. When the concentration of HCl was changed from 2.0 to 10.0 M in the aqueous solution, gold recovery efficiencies, based on the CB[6].HAuCl₄ co-precipitates, decreased from 96.4% to 3.3%. These results indicate that higher concentrations of HCl will lead to the higher solubilities of the CB[6].HAuCl₄ adducts and lower gold recovery efficiencies. Gold recovery efficiency based on CB[6].KAuCl₄ co-precipitates showed (FIG. 4B and Table 7) a similar trend to that of CB[6].HAuCl₄. Upon increasing the concentrations of HCl from 1.0 to 10.0 M, the mixture with 2.0 M HCl exhibited the highest gold recovery efficiency of 96.9%. On basis of these profiles, it can be concluded that the aqueous 2.0 M HCl solutions constitute the optimum solvent system for gold recovery. Gold recovery efficiencies, based on the CB[6].MAuCl₄ co-precipitates, all show a downward trend on increasing or decreasing the concentration of HCl.

TABLE 6
The Gold Recovery Efficiency Changes with the Concentration of HCl, When the
Concentrations of CB[6] and HAuCl₄ in All the Aqueous Solutions are 6 mM

| Concentration of | Au | Au | Au | Avg. | Au in | Total | Recovery |
|---|---|---|---|---|---|---|---|
| HCl/M | 267.595/ppm | 208.209/ppm | 242.795/ppm | Au/ppm | Filtrate/mg | Au/mg | Efficiency |
| 1.0 | 22.700 | 22.571 | 23.290 | 22.854 | 0.571 | 0.591 | 0.033 |
| 2.0 | 19.995 | 19.822 | 20.464 | 20.094 | 0.502 | 0.591 | 0.150 |
| 4.0 | 16.117 | 16.049 | 16.571 | 16.246 | 0.406 | 0.591 | 0.313 |
| 6.0 | 6.781 | 6.938 | 6.900 | 6.873 | 0.172 | 0.591 | 0.709 |
| 8.0 | 0.829 | 0.865 | 0.861 | 0.852 | 0.021 | 0.591 | 0.964 |
| 10.0 | 2.702 | 2.777 | 2.754 | 2.744 | 0.069 | 0.591 | 0.884 |

TABLE 7
The Gold Recovery Efficiency Changes with the Concentration of HCl, When the
Concentrations of CB[6] and KAuCl₄ in All the Aqueous Solutions are 6 mM

| Concentration of | Au | Au | Au | Avg. | Au in | Total | Recovery |
|---|---|---|---|---|---|---|---|
| HCl/M | 267.595/ppm | 208.209/ppm | 242.795/ppm | Au/ppm | Filtrate/mg | Au/mg | Efficiency |
| 1.0 | 22.083 | 21.843 | 23.144 | 22.357 | 0.559 | 0.591 | 0.054 |
| 2.0 | 21.544 | 21.330 | 22.516 | 21.797 | 0.545 | 0.591 | 0.078 |
| 4.0 | 16.815 | 16.626 | 17.546 | 16.996 | 0.425 | 0.591 | 0.281 |
| 6.0 | 7.750 | 7.674 | 8.077 | 7.834 | 0.196 | 0.591 | 0.669 |
| 8.0 | 0.628 | 0.878 | 0.689 | 0.732 | 0.018 | 0.591 | 0.969 |
| 10.0 | 3.233 | 3.268 | 3.398 | 3.299 | 0.082 | 0.591 | 0.860 |

High quality crystals for all five adducts—namely, CB[6].HAuCl₄, CB[6].KAuCl₄, CB[6].HAuBr₄, CB[6]KAuBr₄ and CB[6].HAuCl_{2.28}Br_{1.72}—between CB[6] and MAuX₄, suitable for X-ray crystallography, were obtained by slow liquid-liquid diffusion. When equimolar amounts of HAuCl₄, KAuCl₄, HAuBr₄, KAuBr₄ aqueous solution were layered carefully on the top of aqueous solution of CB[6] containing HCl or HBr, the high quality yellow or brown co-crystals were obtained as a result of the slow mixing of MAuX₄ and CB[6] molecules.

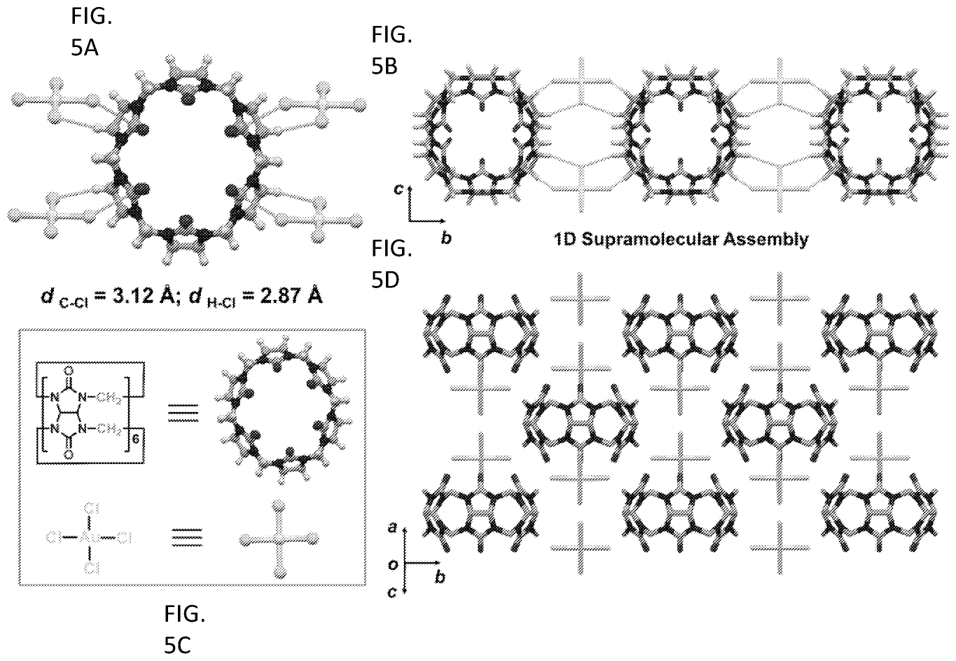

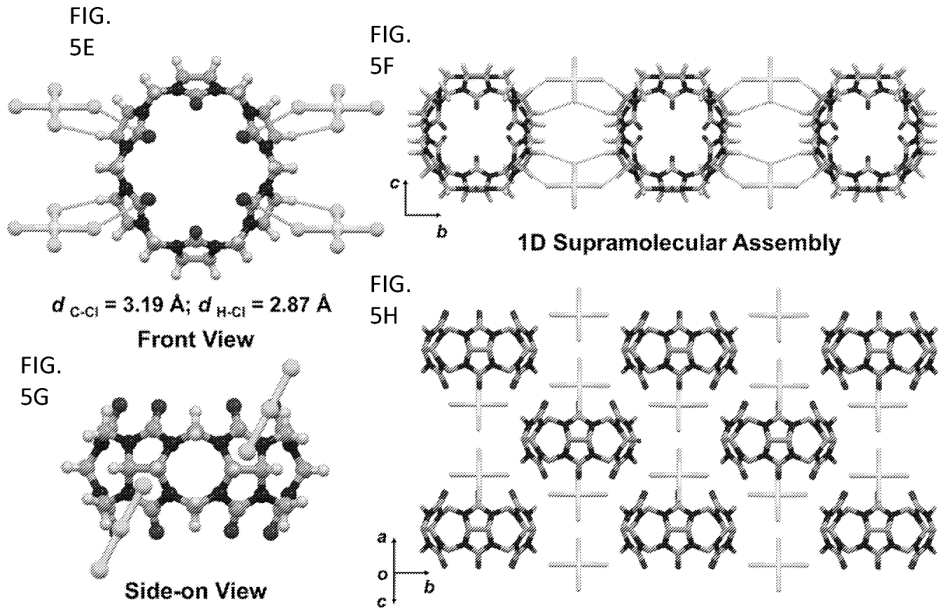

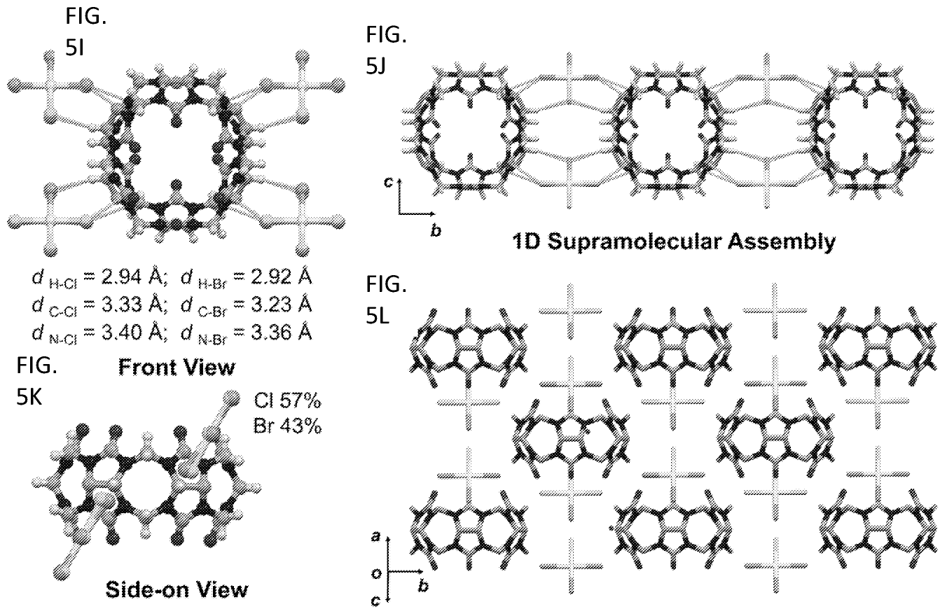

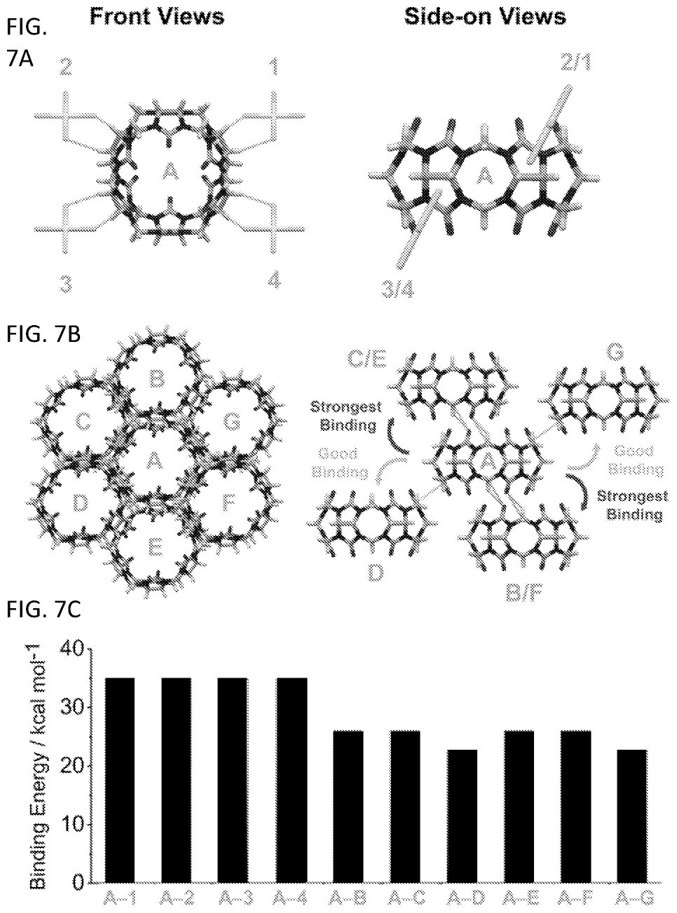

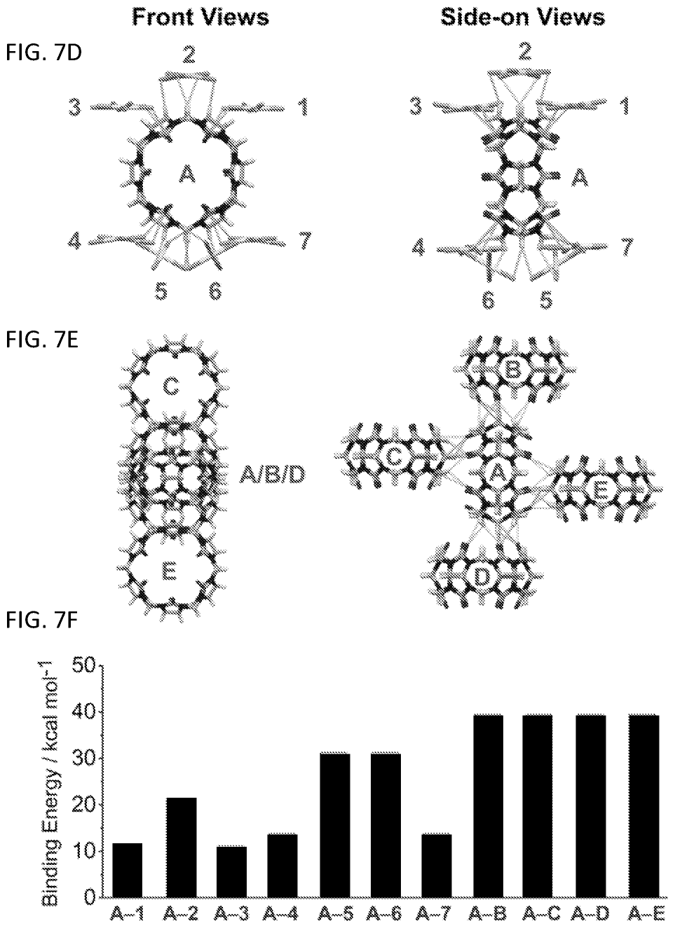

The solid-state superstructure of CB[6].HAuCl₄ reveals (FIGS. 5A-5D and Table 8) that the adduct adopts the monoclinic space group C2/m with β=93.083(2)°, in which every CB[6] molecule interacts (FIG. 5A) with four chemically equivalent [AuCl₄]⁻ anions by the means of hydrogen bonding and ion-dipole interactions. The hydrogen bonding distance in [Au—Cl . . . H—C] involving the Cl atoms in [AuCl₄]⁻ anions and electrostatically positive methine hydrogen atoms on the outer surface of CB[6], is 2.87 Å. The [Au—Cl . . . C═O] ion-dipole interactions refer to the Cl atoms in [AuCl₄]⁻ anions and the portal carbonyl carbon atoms in CB[6], which interact at a distance of 3.12 Å. On the other hand, every CB[6] molecule is surrounded (FIG. 7B) by another six CB[6] molecules. Wherein (i) four of them are sustained by hydrogen bonding and dipole-dipole interactions between the electron-rich carbonyl oxygen atoms on CB[6] and the electron-poor methylene hydrogen and carbonyl carbon atoms on neighboring CB[6] molecules with distances of 2.49 (C═O . . . H—C) and 3.15 (C═O . . . C) Å, respectively. (ii) While the other two CB[6] molecules are held together by the van der Waals interactions with two portal carbonyl carbon atoms at a distance of 3.37 Å. Along the b axis, two adjacent CB[6] molecules are seen (FIG. 5B) to be held together by two parallelly aligned [AuCl₄]⁻ anions at a distance of 9.17 Å (Au . . . Au). Only three of the Cl atoms in the [AuCl₄]⁻ anions exhibit noncovalent bonding with adjacent CB[6] molecules by means of [Au—Cl . . . H—C] and [Au—Cl . . . C═O] interactions. Accordingly, CB[6] molecules and [AuCl₄]⁻ anions adopt an alternate arrangement along the b axis, forming double-connected one dimensional (1D) supramolecular polymers (FIG. 5B). Bundles of these nanostructures are then tightly packed by means of hydrogen bonding and van der Waals interactions between contiguous CB[6] molecules to form (FIG. 5D) a well-ordered array that constitutes the single crystal. X-Ray crystallography reveals (Table 8) that the CB[6].KAuCl₄ adduct has quite similar unit cell parameters compared with that of CB[6].HAuCl₄ adduct. In its solid-state superstructure, CB[6] molecules and [AuCl₄]⁻ anions are also arranged in an alternating manner, forming 1D doubly connected supramolecular polymers (FIGS. 5E-5H), wherein the [AuCl₄]⁻ anions act as linkers between two adjacent CB[6] molecules along the b axis by means of [Au—Cl . . . H—C] hydrogen bonding and [Au—Cl . . . C═O] interactions. Notably, the K⁺ ions are absent in the crystal lattice. This phenomenon indicates that CB[6] crystallize selectively with [AuCl₄]⁻ anions without influence by the counter cations under current experiment condition, i.e., equimolar amounts of CB[6] and KAuCl₄ dissolved in the aqueous HCl (2 M) solution. The example of CB[6], however, interacting preferentially with anions rather than with cations is rare, since the portal carbonyl oxygen atoms in cucurbituril show generally strong affinities with metal and organic cations according to the literature.^{[33}] The possible reasons for the selective crystallization between CB[6] molecules and [AuCl₄]⁻ anions are (i) the K⁺ ions are replaced by protons in aqueous acid solution during the crystallization process, (ii) the CB[6] [AuCl₄]⁻ adducts have the property to be highly crystalline. These fact that the capture of [AuCl₄]⁻ anions with CB[6] molecules to form CB[6].MAuCl₄ adducts is a selective process even in presence of other metal counter cations, which augurs well for developing environmentally benign procedures for the separation of gold from complex mixtures of metal salts.

TABLE 8
Crystallographic Data for Five Complexes Between CB[6] and MAuX₄ (M = H/K, X = Cl/Br)

| Complex | CB[6]•HAuCl₄ | CB[6]•HAuBr₄ | CB[6]•KAuCl₄ | CB[6]•KAuBr₄ | CB[6]•HAuCl_{2.28}Br_{1.72} |
|---|---|---|---|---|---|
| Empirical | C₁₈H₃₂AuCl₄N₁₂O₁₃ | C₇₂H₇₄Au₂Br₈N₄₈O₂₅ | C₃₆H₆₄Au₂Cl₈N₂₄O₂₆ | C₃₆H₃₆AuBr₄K_{0.5}N₂₄O_{12.5} | C₃₆H₆₄Au₂Br_{3.43}Cl_{4.57}N₂₄O₂₆ |
| formula | | | | | |
| Formula | 963.32 | 3045.00 | 1926.64 | 1541.04 | 2079.14 |
| weight | | | | | |
| T/K | 101(2) | 100.00(10) | 100.01(10) | 100.01(12) | 100.00(10) |
| Crystal system | monoclinic | tetragonal | monoclinic | triclinic | monoclinic |
| Space group | C2/m | I4̅2d | C2/m | P1̅ | C2/m |
| a/αα | 16.1427(4) | 14.4358(2) | 16.1569(3) | 14.3299(14) | 16.2232(3) |
| b/Å | 16.3995(4) | 14.4358(2) | 16.4325(3) | 14.3772(11) | 16.5456(3) |
| c/Å | 12.6960(3) | 58.4604(11) | 12.6862(3) | 15.9853(12) | 12.6850(2) |
| α/° | 90 | 90 | 90 | 66.228(7) | 90 |
| β/° | 93.083(2) | 90 | 93.273(2) | 88.468(7) | 93.6000(16) |
| γ/° | 90 | 90 | 90 | 89.781(7) | 90 |
| V/Å³ | 3356.18(14) | 12182.7(4) | 3362.67(12) | 3012.8(5) | 3398.25(10) |
| Z | 4 | 4 | 2 | 2 | 2 |
| ρ_{calcd}/g cm⁻³ | 1.906 | 1.660 | 1.903 | 1.699 | 2.032 |
| μ/mm⁻¹ | 4.779 | 5.110 | 4.770 | 5.200 | 6.601 |
| F (000) | 1900 | 5920 | 1900 | 1497 | 2023 |
| goodness-of- | 1.075 | 1.016 | 1.051 | 1.410 | 1.080 |
| fit on F² | | | | | |
| R₁ [I > 2σ(I)] | 0.0244 | 0.0901 | 0.0334 | 0.1468 | 0.0363 |
| wR₂ [all data] | 0.0634 | 0.2663 | 0.0896 | 0.4354 | 0.0882 |

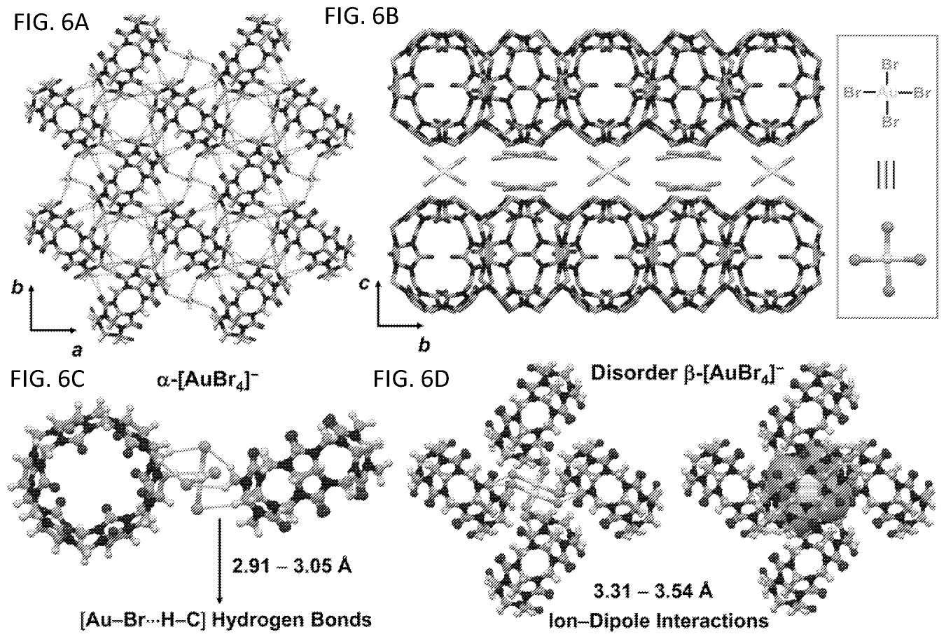

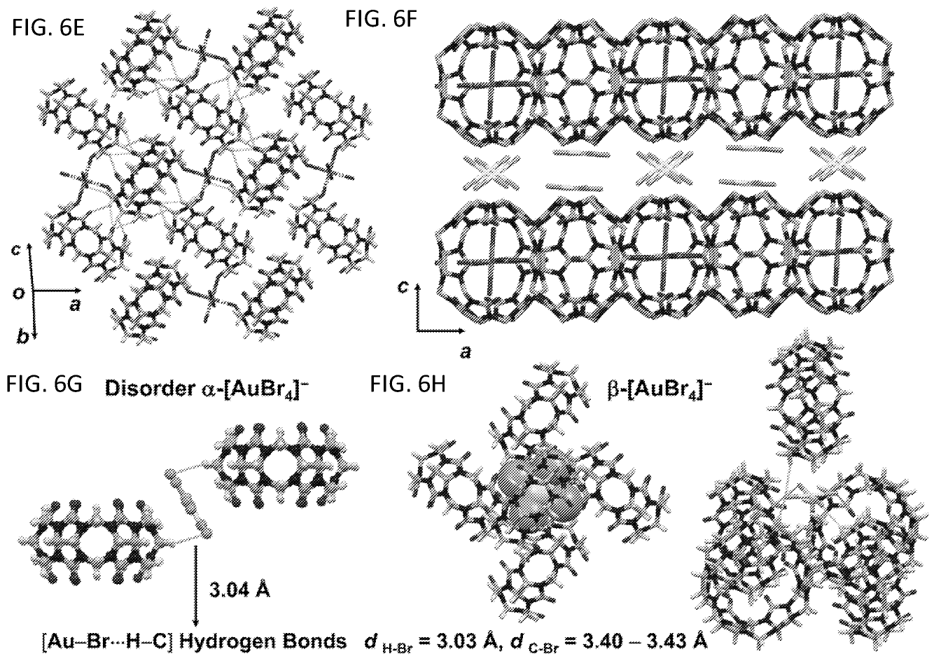

Although the [AuBr₄]⁻ anions possesses a square-planar geometry similar to that of [AuCl₄]⁻ anions, the solid-state superstructure of CB[6].HAuCl₄ is quite different from that of CB[6].HAuCl₄, in which CB[6].HAuCl₄ adopts a tetragonal space group I4̅2d (FIGS. 6A-6D and Table 8). A detailed superstructural analysis displays (FIG. 6A) that, with the presence of water molecules, CB[6] molecules configure a 2D layered superstructure in the a-b plane. The water molecules are disordered and surrounded by four CB[6] molecules courtesy of [C═O . . . H—O] hydrogen bonding with a distance of 2.13 Å. In the 2D layered nanostructure, CB[6] molecules are located perpendicularly to the a-b plane and interact with another four CB[6] molecules sustained (FIG. 7E) by the multiple [C═O . . . H—C] hydrogen bonding and [C═O . . . C] dipole—dipole interactions with distances of 2.23-2.60 and 2.94-3.13 Å, respectively. The 2 D layered nanostructures are observed (FIG. 6B) to be held together by [AuBr₄]⁻ anions relying on C—H hydrogen bonding and ion-dipole interactions to form well-ordered array along the c axis. The [AuBr₄]⁻ anions exhibit two different bonding modes with CB[6] molecules, which will be identified by the descriptors α-[AuBr₄]⁻ and β-[AuBr₄]⁻. The α-[AuBr₄]⁻ anion is related by a 42.8 dihedral angle about the a—b plane and is connected (FIG. 6C) with two CB[6] molecules which are located in adjacent layers as a result of [Au—Br . . . H—C] interactions between Br atoms and both methane and methylene hydrogen atoms on the outer surface of CB[6] with distances ranging from 2.91 to 3.05 Å. The disordered β-[AuBr₄]⁻ anion adopts a parallel arrangement (FIG. 6D) in the a—b plane and interacts with five CB[6] molecules by means of the [Au—Br . . . H—C] hydrogen bonding and [Au—Br . . . C═O] interactions with the distances of 3.04 and 3.31-3.54 Å, respectively. Notably, when an HAuBr₄ aqueous solution was diffused into a CB[6] aqueous solution with HCl (3 M), the brown single crystals were obtained after 12 h. X-Ray crystallographic analysis reveals (FIG. 5K) that 57% of the Br atoms have been exchanged with Cl atoms. The solid-state superstructure (FIGS. SI-5L) of the CB[6].HAuCl_{2.28}Br_{1.72} adduct is quite different from that of CB[6].HAuBr₄, which is isostructural with that of CB[6].HAuCl₄. These results indicate that (i) CB[6].HAuCl_{2.28}Br_{1.72} has good crystallinity, and (ii) the affinity between CB[6] molecules and [AuCl₄]⁻ anions is larger than that between CB[6] molecules and [AuBr4]- anions. In the case of the CB[6].KAuBr₄ adduct, it crystalizes in the triclinic space group P1̅ (FIGS. 6E-6H and Table 8), which is different from that (I4̅2d) associated with the CB[6].HAuCl₄ adduct. In the solid-state superstructure of CB[6].KAuBr₄, the K⁺ ions co-crystallize with the CB[6].[AuBr₄]⁻ adduct in a ratio of 1:2, indicating half of the K⁺ ions take part in crystallization process. This situation differs from the solid-state superstructure of CB[6].KAuCl₄ adduct, in which K⁺ ions are absent. A detailed solid-state superstructural analysis demonstrates that CB[6].KAuBr₄ expresses a similar assembly mode to that of CB[6].HAuBr₄ although they reside in different space groups. In the solid-state superstructure of CB[6].KAuBr₄, CB[6] molecules form (FIG. 5E) a 2D layered superstructure in the a-b plane stabilized by [C=OH—C]interactions between adjacent CB[6] molecules, as well as the ion-dipole interactions involving K⁺ ions and carbonyl oxygen atoms in CB[6], where the K⁺—O distances are in the range of 2.77-2.82 Å. The [AuBr₄]⁻ anions is also accommodated (Figure S2b) in the lattice between two layers, serving as a linker to facilitate a well-order stack of the 2D layered nanostructure. The [AuBr₄]⁻ anions also exhibites (FIG. 5F) two different bonding modes with the CB[6] molecule in the solid-state superstructure of CB[6].KAuBr₄. The α-[AuBr₄]⁻ anion connects (FIG. 5G) with two CB[6] molecules in adjacent layers by means of [Au—Br . . . H—C] interactions with a distance of 3.04 Å. The β-[AuBr₄]⁻ anion interacts (FIG. 5H) with five CB[6] molecules involving the Br atoms of the [AuBr₄]⁻ anions and one methylene hydrogen and four carbon atoms in carbonyl groups with distances in the range of 3.03 and 3.43 Å, respectively. In contrast with the [AuBr₄]⁻ anions in the solid-state superstructure of CB[6].HAuBr₄, the α-[AuBr₄]⁻ anions in the solid-state superstructure of CB[6].KAuBr₄ are disordered (FIGS. 5E-5H) over two positions with 50:50 occupancies, while the β-[AuBr₄]⁻ anions exhibit no disorder. Comparing the solid-state superstructures of CB[6].KAuBr₄ and CB[6].HAuBr₄, it can be found that (i) the H₂O molecules in the 2 D layered superstructure are replaced by K⁺ ions; and (ii) the bonding mode between CB[6] molecules and [AuBr₄]⁻ anions are different.

The solid-state superstructures of the five adducts formed between CB[6] and MAuX₄(M=H/K, Cl/Br) lead us to conclude that (i) both [Aucl₄]⁻ and [AuBr₄]⁻ anions are accommodated in outside instead of inside the cavity of CB[6] aided and abetted by weak hydrogen bonding and ion-dipole interactions between halogen atoms and the electrostatically positive methine, bridged methylene hydrogen atoms and carbonyl carbon atoms on the outer surface of CB[6]; and (ii) that K³⁰ ions provide insignificant contributions to the formation and stabilization of the superstructure, and (iii) although the [AuCl₄]⁻ anion has a similar square-planar geometry to that of the [AuBr₄]⁻ anion, the solid-state superstructures of the CB[6].HAuCl₄ adducts are entirely different from that of CB[6].HAuBr₄.

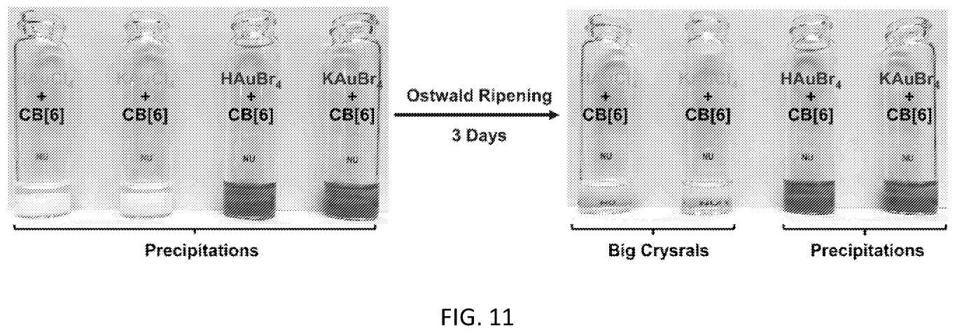

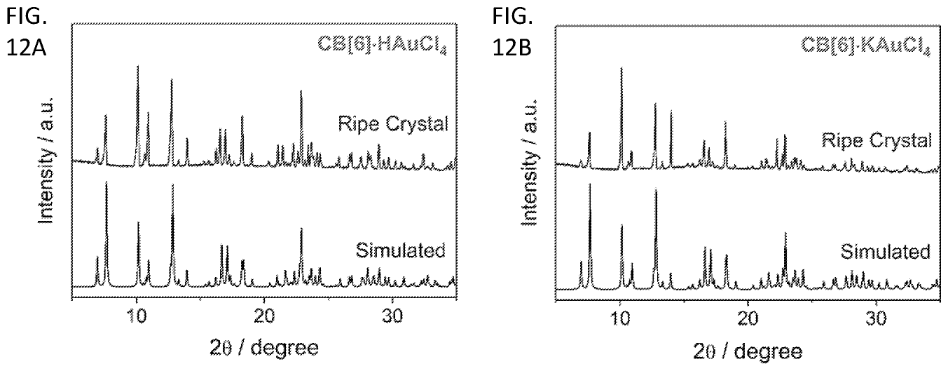

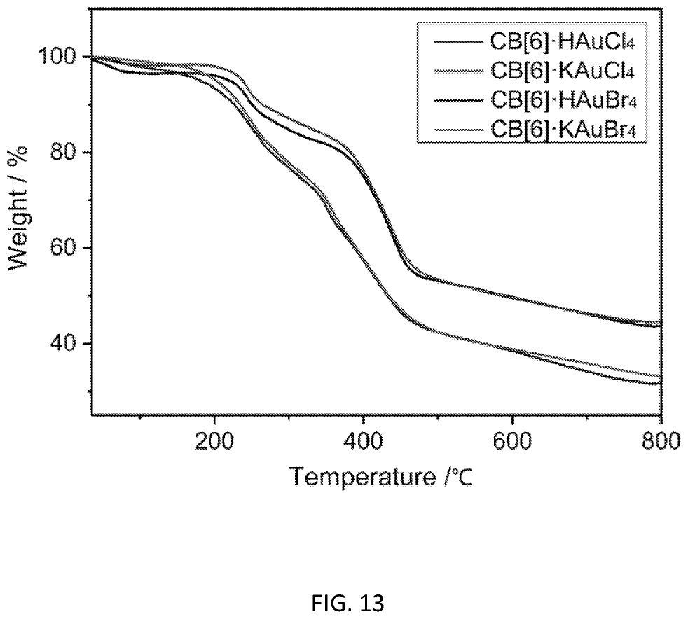

In order to investigate the crystallinity and stability of the four co-precipitates, CB[6].HAuCl₄, CB[6].KAuCl₄, CB[6].HAuBr₄ and CB[6].KAuBr₄, powder X-ray diffraction (PXRD) and thermogravimetric analyses (TGA) were carried out on them. Upon mixing equimolar amounts of CB[6] and MAuX₄(M=H/K, Cl/Br) in aqueous solution with HCl (2 M) or HBr (2.5 M), the yellow or brown suspensions form immediately. All the suspended solids which settled at the bottom of vials after one hour, were subjected to powder XRD analyses after removing supernatant. The experimental PXRD patterns of CB[6].MAuCl₄(M=H/K) matched (FIGS. 10A-10B) well with the simulated patterns based on the single-crystal X-ray data, indicating that the microstructures of the CB[6].MAuCl₄ co-precipitates are consistent with those of crystal superstructures. By contrast, the experimental PXRD patterns of CB[6].MAuBr₄(M=H/K) show (FIGS. 10A-10B) some broad diffraction peaks, an observation which suggests that the co-precipitate of CB[6].MAuBr₄ possesses a weak tendency to crystallize. Interestingly, the co-precipitates of CB[6].MAuCl₄ transform spontaneously to large crystals visible to the naked eye after standing for three days, while the co-precipitates of CB[6].MAuBr₄ show no obvious changes (FIG. 11). This phenomenon demonstrates the fact that co-precipitates of CB[6].MAuCl₄ undergo an Ostwald ripening process^{[34]}. The PXRD patterns for the large crystals (FIGS. 12A-12B) of CB[6].MAuCl₄ are identical with those of the initial co-precipitates, indicating there are no crystalline transformations in Ostwald ripening process. Based on the PXRD analysis of the four adducts, it can be included that the co-precipitate of CB[6].MAuCl₄ is easier to crystallize than that for CB[6].MAuBr₄. The TGA traces reveal (FIG. 13) the fact that the decomposition temperatures for the CB[6].HAuCl₄ (M=H/K) adducts are significantly higher than those for CB[6].MAuBr₄ (M=H/K), suggesting that supramolecular association between CB[6] and MAuCl₄ are more stable than those related to CB[6] and MAuBr₄.

In order to gain better understanding of the different crystallization behavior and the binding energy between CB[6] and HAuX₄ (X=Cl/Br), DFT calculations were carried out based on the solid-state superstructures of the CB[6].HAuCl₄ and CB[6].HAuBr₄ adducts. X-Ray crystallographic analysis of CB[6].HAuCl₄, the central CB[6] molecule is surrounded by six neighboring CB[6] molecules (FIG. 7B). Calculations reveal that the binding energy between the central CB[6] molecules and four identical CB[6] molecules, sustained by the [C═O . . . H—C] interactions, is 26.0 kcal mol⁻¹. This value is higher than that (22.3 kcal mol⁻¹ ) of another two CB[6] molecules that are stabilized by van der Waals interactions (FIG. 7C and Table 9). CB[6] molecules also interact with four equivalent [AuCl₄]⁻ anions (FIG. 7A), the binding energy between CB[6] and them is 34.8 kcal mol⁻¹ (FIG. 7C and Table 10), which is higher than the largest binding energy (26.0 kcal mol⁻¹) between two CB[6] molecules. Herein, most likely, lies the origin as to why the CB[6] is inclined to interact preferentially with [AuCl₄]⁻ anions rather than self-aggregate during the crystallization and precipitate processes. In the case of CB[6].HAuBr₄, every CB[6] molecules interacts with four adjacent CB[6] molecules in the a-b plane (FIG. 7E), and the binding energy between two neighboring CB[6] is 39.5 kcal mol⁻¹ (FIG. 7F and Table 12). While, CB[6] is also surrounded by seven [AuBr₄]⁻ anions stabilized by hydrogen bonding and ion-dipole interactions (FIG. 7D). The binding energy between CB[6] molecules and [AuBr₄]⁻ anions range from 11.3 to 31.3 kcal mol⁻¹ (FIG. 7Fand Table 11). Apparently, binding energy (39.5 kcal mol⁻¹) between two CB[6] molecules is much higher than that between CB[6] molecules and [AuBr₄]⁻ anions, so that the CB[6] has a preference to form (FIG. 6A) a 2D layered nanostructure with itself in the a-b plane. When comparing the binding energy between CB[6] molecules and [AuCl₄]⁻ anions with that of CB[6] molecules and [AuBr₄]⁻ anions, it can be seen (FIGS. 7C and 7F) that the binding energy (34.8 kcal mol⁻¹) of CB[6] molecules to [AuCl₄]⁻ anions is higher than that (11.3-31.3 kcal mol⁻¹) of CB[6] molecules to [AuBr₄]⁻ anions. The bottom line is that the CB[6].MAuCl₄ adducts exhibit obviously better crystallinity and stability than those for CB[6].MAuBr₄, indicating that the [AuCl₄]⁻ anion should be the better candidate when it comes to gold recovery.

TABLE 9
Results of DFT Calculation for the Binding Energies Between
CB[6] (A) and the Six Connected CB[6] (B-G)
Based on the Solid-State Superstructure of CB[6]•HAuCl₄

| Entry | E_{SCF}/ha | E_{vdW}/ha | E_{Total}/ha | E_{b}/kcal mol⁻¹ | E_{b} (%) | Dipole/Debye |
|---|---|---|---|---|---|---|
| A-B | −7211.018 | −0.661 | −7211.679 | 26.028 | 100 | 0.00019 |
| A-C | −7211.018 | −0.661 | −7211.679 | 26.028 | 100 | 0.00021 |
| A-D | −7211.009 | −0.665 | −7211.673 | 22.622 | 87 | 0.00005 |
| A-E | −7211.018 | −0.661 | −7211.679 | 26.028 | 100 | 0.00025 |
| A-F | −7211.018 | −0.661 | −7211.679 | 26.028 | 100 | 0.00026 |
| A-G | −7211.009 | −0.665 | −7211.673 | 22.622 | 87 | 0.00029 |

A-X (X = B-G) represent two interacting molecules named in FIG. 7B.

TABLE 10
Results of DFT Calculation for the Binding Energies Between
CB[6] (A) and the Four Connected [AuCl₄]⁻ Anions
(1-4) Based on the Solid-State Superstructure of CB[6]•HAuCl₄

| Entry | E_{SCF}/ha | E_{vdW}/ha | E_{Total}/ha | E_{b}/kcal mol⁻¹ | E_{b} (%) | Dipole/Debye |
|---|---|---|---|---|---|---|
| A-1 | −5581.229 | −0.353 | −5581.582 | 34.828 | 100 | 17.090 |
| A-2 | −5581.229 | −0.353 | −5581.582 | 34.829 | 100 | 17.084 |
| A-3 | −5581.229 | −0.353 | −5581.582 | 34.829 | 100 | 17.086 |
| A-4 | −5581.229 | −0.353 | −5581.582 | 34.829 | 100 | 17.082 |

A-X (X = 1-4) represent two interacting molecules named in FIG. 7A.

TABLE 11
Results of DFT Calculation for the Binding Energies Between
CB[6] (A) and the Seven Connected [AuBr₄]⁻ Anions
(1-7) Based on the Solid-State Superstructure of CB[6]•HAuBr₄

| Entry | E_{SCF}/ha | E_{vdW}/ha | E_{Total}/ha | E_{b}/kcal mol⁻¹ | E_{b} (%) | Dipole/Debye |
|---|---|---|---|---|---|---|
| A-1 | −14036.109 | −0.358 | −14036.467 | 11.260 | 36 | 10.335 |
| A-2 | −14036.118 | −0.364 | −14036.483 | 21.278 | 68 | 13.960 |
| A-3 | −14036.109 | −0.358 | −14036.467 | 11.234 | 36 | 10.329 |
| A-4 | −14036.110 | −0.361 | −14036.471 | 13.832 | 44 | 10.068 |
| A-5 | −14036.109 | −0.370 | −14036.479 | 31.252 | 100 | 10.321 |
| A-6 | −14036.109 | −0.370 | −14036.479 | 31.266 | 100 | 10.379 |
| A-7 | −14036.110 | −0.361 | −14036.471 | 13.832 | 44 | 10.070 |

A-X (X = 1-7) represent two interacting molecules named in FIG. 7D.

TABLE 12
Results of DFT Calculation for the Binding Energies Between CB[6] (A) and Four
Connected CB[6] (B-E) Based on the Solid-State Superstructure of CB[6]•HAuBr₄

| Entry | E_{SCF}/ha | E_{vdW}/ha | E_{Total}/ha | E_{b}/kcal mol⁻¹ | E_{b} (%) | Dipole/Debye |
|---|---|---|---|---|---|---|
| A-B | −3605.433 | −0.324 | −3605.757 | 39.481 | 100 | 7.976 |
| A-C | −7210.866 | −0.648 | −7211.515 | 39.479 | 100 | 7.975 |
| A-D | −3605.433 | −0.324 | −3605.757 | 39.481 | 100 | 7.976 |
| A-E | −7210.866 | −0.648 | −7211.515 | 39.479 | 100 | 7.975 |

A-X (X = B-E) represent two interacting molecules named in FIG. 7E.

In an attempt to test the validity of CB[6] for gold recovery, a gold-bearing alloy wire was employed as a model of gold-bearing scrap, to develop a laboratory-scale gold recovery process (FIG. 8B). On account of the highest co-precipitation yield (99.2%) and good crystallization properties of the CB[6].HAuCl₄ adduct, HAuCl₄ was chosen as the intermediate to test. A yellow gold-bearing alloy wire, containing 58% wt of Au and 42% wt of Cu, Zn and Ag, was etched by the least amount of a mixture of conc HCl and HNO₃, wherein Au is converted into HAuCl₄. According to the prior optimization experiments, the acid concentration of HAuCl₄-containing mixture solution was diluted to 2 M. Insoluble AgCl impurities were removed by filtration. When the addition of an aqueous solution of CB[6] to the residual filtrate, the co-precipitation of CB[6].HAuCl₄ occurred immediately. This phenomenon indicates that such a large amounts of Cu, Zn and Ag salts had negligible impact on the co-precipitation process. After filtration of the co-precipitates from the mixture, the CB[6].HAuCl₄ co-precipitate was dispersed in aqueous acid solution and reduced with N₂H₄.H₂O. Finally, the gold was recovered by filtration. ICP-OES elements analysis reveals (Table 13) that 99.8% of gold in raw material was recovered. In addition, the remaining HAuCl₄ in filtrate can be recycled, while the CB[6] can be reused after recrystallization, improving significantly the utilization of reagents and decreasing the recovery costs. This laboratory-scale gold recovery process provides us with a new way of developing a highly efficient, feasible and environmentally friendly process for industrial production, as well as avoiding the use of the hypertoxic cyanide and mercury in traditional gold mining practices all around the world.

TABLE 13
The Gold Recovery Efficiency from Gold Alloy Based on the Co-precipitate of CB[6]•HAuCl₄

| | Au | Au | Au | Avg. | Au in | Total | Recovery |
|---|---|---|---|---|---|---|---|
| | 267.595/ppm | 208.209/ppm | 242.795/ppm | Au/ppm | Filtrate/mg | Au/mg | Efficiency |
| CB[6]•Au Alloy | 0.168 | 0.157 | 0.184 | 0.170 | 0.005 | 2.403 | 0.998 |

TABLE 14
The Purity of Recovered Gold

| | Au | Au | Au | Avg. | Au in | Recovered | |
|---|---|---|---|---|---|---|---|
| | 267.595/ppm | 208.209/ppm | 242.795/ppm | Au/ppm | solution/mg | Au/mg | Purity |
| Recovered | 0.234 | 0.234 | 0.243 | 0.237 | 1.423 | 1.450 | 0.981 |
| Gold | | | | | | | |

In conclusion, an instantaneous self-assembly of CB[6] molecules and MAuX₄ anions (M=H/K, X=Cl/Br) leads to rapid co-precipitation of CB[6].HAuCl₄, CB[6]KAuCl₄, CB[6].HAuBr₄, and CB[6]KAuBr₄ adducts. During the systematic optimization of the conditions, we found that gold recovery efficiencies, based on the co-precipitations, are concentration-dependent, involving CB[6], MAuX₄ and acid. The higher the initial concentration of CB[6] and MAuX₄, the better gold recovery efficiency. The CB[6]HAuCl₄ adduct affords the highest yield (99.2%) for the gold recovery. The aqueous 2 M HCl solution was confirmed to be the most suitable solvent system for this gold recovery process. Single crystal structures for all the four adducts revealed that the weak [Au—X . . . H—C] hydrogen bonding and [Au—X . . . C═O] ion-dipole interactions be the main driving forces for the formation of the co-precipitates between CB[6] and MAuX₄. CB[6] molecules and [AuCl₄]⁻ anions adopt an alternating arrangement in the crystal superstructure, while [AuBr₄]⁻ anions accommodated in the lattice between the 2D layered nanostructures made up of CB[6]. The CB[6].MAuCl₄ adducts show better crystallinity and stability than that of CB[6].MAuBr₄, which might be resulted from the higher binding energy between CB[6] molecules and [AuCl₄]⁻ anions than that of CB[6] molecules and [AuBr4]⁻ anions. These fundamental investigations of the multiple non-covalent interactions between CB[6] and MAuX₄, not only provides insight into the nature of the precipitation process, but also indicates that subtle changes in building blocks will lead to different superstructures and properties. In addition, a laboratory-scale gold recovery process was established based on the co-precipitate of CB[6].HAuCl₄, in which 99.8% of gold in raw material was recovered. Such a gold recovery strategy not only leads to a fast, feasible and economic process with a high recovery efficiency, but also is more environmentally friendly in comparison with the universal cyanidation process, which impressively satisfies the requirement for industrial production, demonstrating its great potential in practical application.

### Materials

All gold salt (HAuCl₄, KAuCl₄, HAuBr₄, KAuBr₄), HCl (wt 37%) aqueous solutions and HBr (wt 47-49%) aqueous solutions were purchased from commercial suppliers and used without further purification unless stated otherwise. The cucurbit[6]uril was synthesized according to the previous literature with some modifications¹. High purity water was generated by a Milli-Q.

### General Methods

### Co-Precipitation Experiments of CB[6] with MAuX₄(M=H/K, X=Cl/Br)

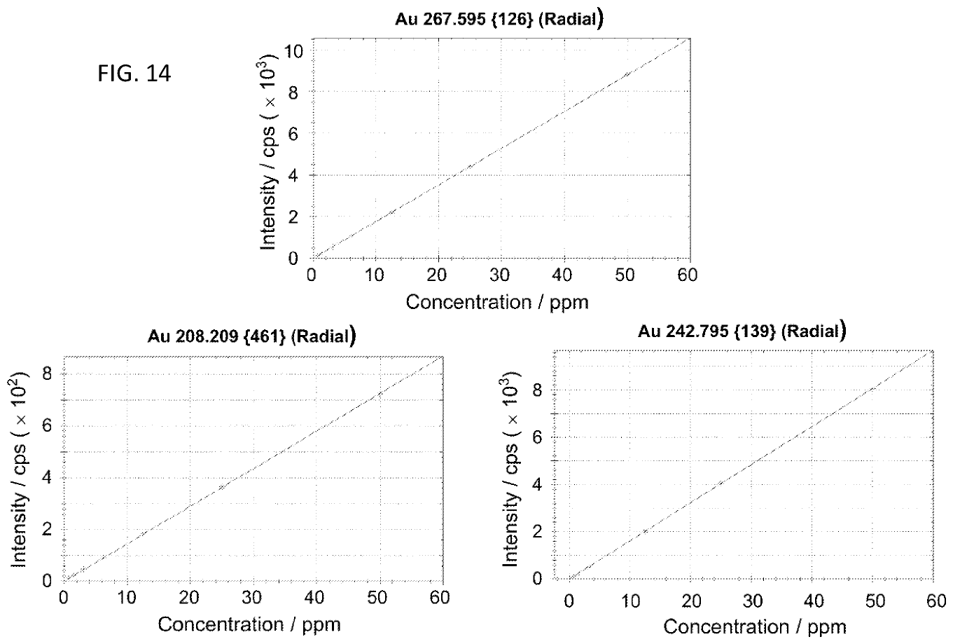

Aqueous stock solutions of HAuCl₄, KAuCl₄, HAuBr₄, KAuBr₄ were prepared by dissolving directly the corresponding commercially available salts in high purity H₂O, while aqueous solutions of CB[6] were prepared by dissolving the CB[6] powder with different concentrations of HCl or HBr. When CB[6] (8 mM) in an aqueous HCl or HBr solution was added to a MAuX₄ (20 mM, M=H/K, X=Cl/Br) aqueous solution, yellow and brown co-precipitates were formed immediately. The yellow and brown solids were isolated by filtration and air-dried. The concentrations of [AuX₄]⁻ (M=Cl/Br) remaining in the filtrates were analyzed by ICP-OES elemental analysis and compared with standard solutions (FIG. 14). The gold recovery efficiencies from the co-precipitation experiments were calculated based on the initial and residue concentrations of [AuX₄]⁻ in the aqueous solution.

### Process for Gold Recovery from Gold-Bearing Materials

A yellow gold-bearing alloy wire, composed of 58% wt Au, 42% wt of Cu, Zn and Ag, was employed (FIG. 8B) as gold-bearing scrap in a laboratory-scale gold recovery experiment. The yellow gold-bearing alloy (30 mg) was first of all etched by a mixture of conc HCl and HNO₃ (3/1, V/V). Subsequently, the concentration of acid in the resulting gold-bearing solution was diluted to ˜2 M with high purity H₂O, and insoluble AgCl impurities were removed by filtration. Upon the addition of an aqueous solution of CB[6] (40 mM) to the residual filtrate, the yellow co- precipitate of CB[6].HAuCl₄, which formed immediately, was separated from other metals by filtration. Finally, the CB[6].HAuCl₄ co-precipitation was dispersed in aqueous solution and then reduced with N₂H₄.H₂O. The gold was recovered by filtration. The residual CB[6] in the solution can be recycled after recrystallization. The ICP-OES elemental analysis revealed (Table 14) that 99.8% of gold in raw material was recovered and its purity is 98.1%.

### Crystallographic Characterization

The crystals for all the five adducts—namely, CB[6].HAuCl₄, CB[6].KAuCl₄, CB[6].HAuBr₄, CB[6].KAuBr₄, CB[6].HAuCl_{2.28}Br_{1.72}—between CB[6] and MAuX₄ which were suitable for single crystal X-ray crystallography, were obtained by slow liquid-liquid diffusion. The detailed procedures were executed as follows. The stock aqueous solutions of HAuCl₄ and KAuCl₄ (20 mM, 100 μL) were layered carefully upon an aqueous CB[6] (10 mM, 200 μL) solution with 3 M HCl in 1-mL tubes. High quality yellow co-crystals of CB[6].HAuCl₄ and CB[6]KAuCl₄ were obtained after about 12 h. Using a similar procedure, aqueous stock solutions of HAuBr₄ and KAuBr₄ (20 mM, 100 μL) were layered carefully upon the aqueous CB[6] (8 mM, 250 μL) solution with 3.5 M HBr in 1-mL tubes. High quality brown co-crystals of CB[6].HAuBr₄ and CB[6].KAuBr₄ were obtained after about 1 day. The aqueous stock solution of HAuBr₄ (20 mM, 100 μL), which was carefully layered onto the aqueous CB[6] (10 mM, 200 μL) solution with 3 M HCl in 1-mL tubes, produced high quality brown co-crystals of CB[6].HAuCl_{2.28}Br_{1.72} after about 12 h.

Suitable crystals were selected and mounted on a MITIGEN holder with Paratone oil on an XtaLAB Synergy, Single source at offset/far, HyPix diffractometer. The crystals were kept at ˜100 K during the data collection. Using Olex2,² the structures were solved with the ShelXT³ structure solution program using Intrinsic Phasing and refined with the XL4 refinement package employing Least Squares Minimization. Crystallographic images were produced using Mercury 4.3.0. Distances and angles were measured employing Mercury 4.3.0. FIGS. 5-7 and FIGS. S1-S3 show the single-crystal packing superstructures of CB[6].HAuCl₄, CB[6].KAuCl₄, CB[6].HAuBr₄, CB[6].KAuBr₄, and CB[6].HAuCl_{2.28}Br_{1.72}, respectively. Table 8 reports the crystallographic data of all five crystalline adducts.

### CB[6]KAuCl₄

(a) Refinement and solvent treatment details. No special refinement was necessary in the case of solving the solid-state superstructure of CB[6]KAuCl₄. The solvent masking procedure, as implemented in Olex2, was used to remove the electronic contributions of solvent molecules from the refinement. As the exact solvent content is not known, only the atoms used in the refinement model are reported in the formula here. Total solvent accessible volume/cell=285.0 Å³[8.5%] and total electron count/cell=51.8. The solid-state (super)structure of CB[6].KAuCl₄ is illustrated in FIGS. 5E-55H.

### CB[6]KAuBr₄

(a) Refinement details. The crystal of CB[6].KAuBr₄ was found to be non-merohedrally twinned. The orientation matrices for the two components were identified using the program CrysAlisPro (Rigaku Oxford Diffraction, 2019). The exact twin matrix, identified by the integration program, was found to be (−1.0008 −0.0002 −0.0005/−0.0007 −1.0006 −0.0034/0.8910 0.0538 0.9990). The second domain is rotated from first domain by 180° about the reciprocal lattice c axis. An hklf5 file was used in all refinements. The twin fraction refined to a value of 0.315(3). Distance restraints were imposed on the C—N bonds. The enhanced rigid-bond restraint (SHELX keyword RIGU) was applied globally.⁵ Restraints on similar amplitudes separated by less than 1.7 Ang. were also imposed globally.

(b) Solvent treatment details. In the case of the crystal of CB[6].KAuBr₄, the disordered solvent molecules could not be modeled adequately. The bypass procedure in Platon was used to remove the electronic contribution from these solvents. The total potential solvent accessible void volume was 573 Å³ and the electron count/cell=152.0. As the exact solvent content is not known, the reported formula reflects only the atoms used in the refinement. The solid-state (super)structure of CB[6].KAuBr₄ is shown in FIGS. 6E-6H.

### CB[6].HAuCl_{2.28}Br_{1.72}

(a) Refinement and solvent treatment details. In the crystal of CB[6].HAuCl_{2.28}Br_{1.72}, chlorine and bromine atoms were disordered individually. The solvent masking procedure, as implemented in Olex2, was used to remove the electronic contribution of the solvent molecules from the refinement. As the exact solvent content is not known, only the atoms used in the refinement model are reported in the formula here. Total solvent accessible volume/cell=283.8 Å³ [8.4%] and total electron count/cell=55.6 The solid-state (super)structure of CB[6].HAuCl_{2.28}Br_{1.72} is illustrated in FIGS. 5I-5L.

### Powder X-Ray Diffraction Analysis

Powder X-ray diffraction (PXRD) analyses were carried out on a STOE-STADI MP powder diffractometer equipped with an asymmetric curved Germanium monochromator (Cu-Kα1 radiation, λ=1.54056 Å) and a one-dimension silicon strip detector (MYTHEN2 1K from DECTRIS). Samples for structural analysis were measured at room temperature in transmission geometry. The simulated PXRD patterns were calculated using the Mercury software 4.3.0.

### Fourier-Transform Infrared/Thermogravimetric Analysis

Fourier-transform infrared (FT-IR) spectroscopy was performed on a Nexus 870 spectrometer (Thermo Nicolet) in the mode of attenuated total reflection (ATR) with the range from 4000 to 600 cm⁻¹ and at a resolution of 0.125 cm⁻¹.

### Thermogravimetric Analysis

Thermogravimetric analysis (TGA) experiments were performed on a Mettler Toledo TGA/DSC I Stare System (Schwerzenbach, Switzerland) interfaced with a PC using Stare software. Samples were placed in an Al₂O₃ crucible and heated at a rate of 10 K min⁻¹ from 35 to 800° C. under a helium atmosphere.

### Density Function Theory Calculations

In order to gain a better understanding of the driving force and bonding energy between CB[6] and [AuCl₄]⁻, [AuBr₄]⁻, as well as the adjacent CB[6] molecules, DFT calculations have been carried out based on the crystal superstructures of CB[6].HAuCl₄ and CB[6].HAuBr₄. FIGS. 7A-7B show the binding modes of CB[6] and [AuCl₄]⁻, as well as those between adjacent CB[6] molecules in the solid-state superstructure of CB[6].HAuCl₄, respectively. Tables 9-10 summarizes the binding energies between CB[6] and [AuCl₄]⁻, as well as those between adjacent CB[6] molecules, according to DFT calculations. FIGS. 7D-7E show the binding modes of CB[6] and [AuBr₄]⁻, as well as those between adjacent CB[6] molecules in the solid-state superstructure of CB[6].HAuBr₄, respectively. Tables 11-12 summarizes the binding energies between CB[6] and [AuBr₄]⁻, as well as those between adjacent CB[6] molecules, according to DFT calculations.

The DFT calculations were performed with the Orca program (version 4.1.0). The hybrid functional Becke three-peramater Lee-Yang-Parr (B3LYP) with Grimme's van der Waals corrections with Beck-Johnson damping (D3BJ) were used. Ahlrich's double zeta basis set with a polarization function (Def2-SVP) and electron-core potentials (Def2-ECPs) were used. The binding energies were calculated from E_{b, AB}=E_{total,A}−E_{total,B}. The resolution of the identity with Coulomb integral and numerical chain-of-sphere integration for the HF exchange (RIJCOSX) was applied to improve the computational efficiency.

The meanings of physical quantity is recorded in the Tables S2-5 are as follows—

E_{SCF} is the converged electronic energy.

E_{vdW} is the van der Waals correction energy.

Eₜₒₜₐᵢ is the van der Waals -corrected total electronic energy.

E_{b} is the binding energy (a thermodynamic quantity).

### ICP-OES Elemental Analysis

Inductively coupled plasma optical emission spectrometry (ICP-OES) was performed on a thermo iCap7600 ICP-OES (Thermo Fisher Scientific, Waltham, Mass., USA) operating in radial view and equipped with a CETAC 520 autosampler (Omaha, Neb., USA). The samples were filtered through a 0.45-μm filter. The filtrates were then diluted with ultrapure H₂O and analyzed for the concentration of Au in comparison with standard solutions. Each sample was recorded using 5 sec visible exposure and 15 sec UV exposure time. Each sample was measured repeatedly for 3 times. The wavelengths selected for the analyses of the concentration of Au were 208.209, 242.795, and 267.595 nm.

### REFERENCES

(1) (a) Kim, J.; Jung, I. S.; Kim, S. Y.; Lee, E.; Kang, J. K.; Sakamoto, S.; Yamaguchi, K.; Kim, K. New Cucurbituril Homologues: Syntheses, Isolation, Characterization, and X-Ray Crystal Structures of Cucurbit[n]uril (n=5, 7, and 8). J. Am. Chem. Soc. 2000, 122, 540-541. (b) Day, A.; Arnold, A. P.; Blanch, R. J.; Snushall, B. Controlling Factors in the Synthesis of Cucurbituril and Its Homologues. J. Org. Chem. 2001, 66, 8094-8100.
(2) Dolomanov, O. V.; Bourhis, L. J.; Gildea, R. J.; Howard, J. A. K.; Puschmann, H. OLEX2: A Complete Structure Solution, Refinement and Analysis Program. J. Appl. Cryst. 2009, 42, 339-341.
(3) Sheldrick, G. M. SHELXT—Integrated Space-Group and Crystal-Structure Determination. Acta. Cryst. 2015, A71, 3-8.

### (4) Sheldrick, G. M. A Short History of SHELX Acta Cryst 2008, A64, 112-122.

(5) Thorn, A. Dittrich, B. Sheldrick, G. M. Enhanced Rigid-Bond Restraints. Acta. Cryst. 2012, A68, 448-451.

1. An adduct comprising a metal halide anion non-covalently bound to the outer surface of a macrocycle.

2. The adduct assembly of claim 1, wherein the macrocycle is a cucurbituril and/or the metal halide anion is [AuX₄]⁻ and X is a halogen.

3. The adduct of claim 2, wherein the macrocycle is cucurbitu[6]ril.

4. The adduct of claim 2, wherein the metal halide anion is [AuX₄]⁻.

5. The adduct of claim 2, wherein the adduct comprises the cucurbituril and [AuX₄]⁻, wherein the metal halide anion is non-covalently bound by a [Au—X . . . H—C] hydrogen bond and/or a [Au—X . . . C═O] ion-dipole interaction.

6. A superstructure comprising the adduct of claim 1.

7. The superstructure of claim 6, wherein the metal halide anion comprises Cl and the superstructure comprises an alternating one-dimensional supramolecular assembly where adjacent macrocycles are connected to two parallelly aligned metal halide anions.

8. The superstructure of claim 7, wherein the superstructure comprises parallelly aligned one-dimensional supramolecular assemblies.

9. The superstructure of claim 6, wherein the metal halide anion comprises Br and the superstructure comprises a two-dimensional supramolecular assembly comprising the macrocycle where the metal halide anion is accommodated between the lattice space between the two-dimensional supramolecular assemblies.

10. A crystalline composition comprising the adduct of claim 1.

11. The crystalline composition of claim 10, wherein the crystalline composition is in the monoclinic space group C2/m and a=16.2±0.2 Å, b=16.4±0.2 Å, c=12.7±0.2 Å, α=90.0±0.5°, β=93.3±0.5°, and γ=90.0±0.5°.

12. The crystalline composition of claim 10, wherein the crystalline composition is in the monoclinic space group I4̅2d and a=14.4±0.2 Å, b=14.4±0.2 Å, c=58.5±0.2 Å, α=90.0±0.5°, β=90.0±0.5°, and γ=90.0±0.5°.

13. The crystalline composition of claim 10, wherein the crystalline composition is in the monoclinic space group P1̅and a=16.2±0.2 Å, b=16.5±0.2 Å, c=12.7±0.2 Å, α=90.0±0.5°, β=93.5±0.5°, and γ=90.0±0.5°.

14. A method for isolating gold from a gold-bearing material, comprising:
(a) contacting the gold-bearing material with a hydrogen halide to form a gold-halide solution;
(b) contacting the gold-halide solution with a macrocycle to form a precipitate, the precipitate comprising the adduct of claim 1; and
(c) isolating the precipitate.

15. The method of claim 14, wherein the method further comprises reducing gold of the precipitate with a reductant.

16. The method of claim 15 further comprising isolating the reduced gold of the precipitate.

17. The method of claim 14, wherein the method further comprises isolating the macrocycle after formation of the adduct.

18. The method of claim 17, wherein the isolated macrocycle is recycled by contacting the isolated macrocycle with the gold-halide solution in step (b).

19. The method of claim 14, wherein contacting the gold-bearing material comprises etching the gold-bearing material with an etchant comprising the hydrogen halide.

20. The method of claim 14, wherein the hydrogen halide is HCl or HBr.

21. (canceled)

22. (canceled)
